(19) United States
(12) Patent Application Publication
(10) Pub. No.: US 2026/0168021 A1
(43) Pub. Date: Jun. 18, 2026

(54) METHODS AND COMPOSITIONS FOR ASSESSING ACTIVITY OF TARGETED CIS-REGULATORY DNA ELEMENTS

(71) Applicant: President and Fellows of Harvard College, Cambridge, MA (US)

(72) Inventors: Brian Bor-Jen Liau, Cambridge, MA (US); Hee Jin Roh, Cambridge, MA (US); Simon Shen, Cambridge, MA (US); Yan Hu, Cambridge, MA (US)

(73) Assignee: President and Fellows of Harvard College, Cambridge, MA (US)

(21) Appl. No.: 19/420,546

(22) Filed: Dec. 15, 2025

Related U.S. Application Data
(60) Provisional application No. 63/734,670, filed on Dec. 16, 2024.

(51) Int. Cl. C12Q 1/6869; C12N 9/22; C12N 15/11; C12Q 1/34

(52) U.S. Cl. CPC C12Q 1/6869; C12N 9/226; C12N 15/111; C12Q 1/34; C12N 2310/20

(57) ABSTRACT

Aspects of the technology relate to methods for identifying a therapeutic genomic target sequence that include perturbing a genomic locus using a gene editing system, deaminating one or more nucleotides of the genomic locus using a deaminase to produce a pattern of deamination at the genomic locus, and identifying a therapeutic genomic target sequence based on the pattern of deamination at the genomic locus.

### CROSS-REFERENCE TO RELATED APPLICATIONS

This application claims the benefit under 35 U.S.C. § 119 (e) of U.S. Provisional Application No. 63/734,670, filed Dec. 16, 2024, which is herein incorporated by reference in its entirety.

### REFERENCE TO AN ELECTRONIC SEQUENCE LISTING

The contents of the electronic sequence listing (H049870850US01-SEQ-KVC.xml; Size: 41,956 bytes; and Date of Creation: Dec. 15, 2025) are herein incorporated by reference in their entirety.

### BACKGROUND

Cis-regulatory elements (CREs) are DNA sequences that act as molecular switches to control gene expression. They frequently occur in dense clusters and comprise finer-scale components that collectively govern transcription factor (TF) binding, chromatin accessibility, and the expression of nearby genes. Chromatin-accessibility profiling has yielded high-resolution insights into CRE function and regulatory architecture, while CRISPR-based genome-editing technologies now enable systematic perturbation and fine-mapping of endogenous CREs. More broadly, understanding how genome editing reshapes chromatin accessibility is important for clarifying the molecular mechanisms that connect genetic alterations to downstream phenotypic outcomes.

### SUMMARY

Genome editing enables sequence-function profiling of endogenous cis-regulatory elements, driving understanding of their mechanisms and the development of gene therapies. However, these approaches cannot be combined with direct scalable readouts of chromatin structure and accessibility across long single-molecule chromatin fibers. Herein, a double-stranded DNA cytosine deaminase is leveraged to profile chromatin accessibility at high depth and resolution at endogenous loci of interest through targeted PCR and long-read sequencing, a method we term targeted deaminase-accessible chromatin sequencing (TDAC-seq). Powered by high sequence coverage at targeted loci of interest, TDAC-seq can be uniquely integrated with CRISPR perturbations to enable the functional dissection of cis-regulatory elements, where genetic perturbations and their effects on chromatin accessibility are superimposed on the same single chromatin fiber and resolved at single-nucleotide resolution. TDAC-seq was employed to parse CRISPR edits that activate fetal hemoglobin in human CD34+ hematopoietic stem and progenitor cells (HSPCs) during erythroid differentiation as well as in pooled CRISPR and base editing screens tiling an enhancer controlling the globin locus. The method was further scaled to interrogate 947 variants in a GF11B-linked enhancer associated with myeloproliferative neoplasm risk in a single pooled CRISPR experiment in CD34⁺ HSPCs. Together, TDAC-seq enables high-resolution sequence-function mapping of, for example, single-molecule chromatin fibers by genome editing.

Some aspects of the technology relate to a method for identifying a therapeutic genomic target sequence, the method comprising: (a) perturbing a genomic locus using a gene editing system; (b) deaminating one or more nucleotides of the genomic locus using a deaminase to produce a pattern of deamination at the genomic locus; and (c) identifying a therapeutic genomic target sequence based on the pattern of deamination at the genomic locus.

Other aspects of the technology relate to a method for screening for guide RNAs targeting regulatory elements that modulate chromatin accessibility, the method comprising: (a) perturbing genomic loci using a CRISPR-Cas system comprising a pooled library of guide RNAs; (b) deaminating one or more nucleotides of the respective genomic loci using a deaminase to produce a pattern of deamination at the respective genomic loci; and (c) identifying guide RNAs targeting regulatory elements that modulate chromatin accessibility based on the pattern of deamination at the respective genomic loci.

In some embodiments, the gene editing system is selected from CRISPR-Cas systems, TALEN (Transcription Activator-Like Effector Nuclease) systems, ZFN (Zinc Finger Nuclease) systems, and meganuclease (homing endonuclease) systems. In some embodiments, the gene editing system is a CRISPR-Cas system. In some embodiments, the CRISPR-Cas system comprises a guide RNA (gRNA) and a Cas nuclease, optionally selected from Cas9 enzymes and Cas12 enzymes. In some embodiments, a guide RNA (gRNA) comprises the nucleotide sequence of any one of SEQ ID NOs: 1-10.

In some embodiments, the CRISPR-Cas system comprises a deactivated Cas (dCas) nuclease. In some embodiments, the CRISPR-Cas system further comprises a base editor, optionally selected from cytosine base editors and adenine base editors.

In some embodiments, the CRISPR-Cas system comprises a Cas nickase (nCas). In some embodiments, the CRISPR-Cas system further comprises a reverse transcriptase.

In some embodiments, the deaminase is a double-stranded DNA deaminase. In some embodiments, the double-stranded DNA deaminase is double-stranded DNA deaminase toxin A (DddA), DddB, DddSs, DddDd-5, MGYFPDa829, or CseDa01, and optionally wherein the DddA is DddA11.

In some embodiments, the genomic locus comprises a cis-regulatory element.

In some embodiments, the method comprises amplifying the genomic locus, optionally using PCR (polymerase chain reaction), to produce amplicons of the genomic locus. In some embodiments, the method comprises half-nested PCR with a long extension time (15 minutes) using KOD One PCR Master Mix to increase the PCR product yield. In some embodiments, the method further comprises sequencing the amplicons of the genomic locus.

In some embodiments, sequencing of the amplicons comprises long-read sequencing, optionally nanopore sequencing or single-molecule real-time sequencing.

In some embodiments, the method of the present disclosure further comprises adding a uracil-DNA glycosylase inhibitor (UGI).

Aspects of the present disclosure relate to a kit, comprising a deaminase, a gene editing system, and materials and/or reagents for executing any method described here.

In some embodiments, the deaminase is a double-stranded DNA deaminase. In some embodiments, the double-stranded DNA deaminase is double-stranded DNA deaminase toxin A (DddA), DddB, DddSs, DddDd-5, MGYFPDa829, or CseDa01. In some embodiments, the DddA is DddA11.

In some embodiments, the gene editing system is selected from CRISPR-Cas systems, TALEN (Transcription Activator-Like Effector Nuclease) systems, ZFN (Zinc Finger Nuclease) systems, and meganuclease (homing endonuclease) systems.

In some embodiments, the gene editing system is a CRISPR-Cas system. In some embodiments, the CRISPR-Cas system comprises a guide RNA (gRNA) and a Cas nuclease, optionally selected from Cas9 enzymes and Cas12 enzymes. In some embodiments, a guide RNA (gRNA) comprises the nucleotide sequence of any one of SEQ ID NOs: 1-10. In some embodiments, the CRISPR-Cas system comprises a deactivated Cas (dCas) nuclease. In some embodiments, the CRISPR-Cas system further comprises a base editor, optionally selected from cytosine base editors and adenine base editors. In some embodiments, the CRISPR-Cas system comprises a Cas nickase (nCas). In some embodiments, the CRISPR-Cas system further comprises a reverse transcriptase.

In some embodiments, the kit further comprises a uracil-DNA glycosylase inhibitor (UGI).

### BRIEF DESCRIPTION OF THE DRAWINGS

FIGS. 1A-1G. TDAC-seq detects chromatin accessibility across kilobase regions through targeted PCR. (FIG. 1A) Schematic of TDAC-seq to map chromatin accessibility along targeted genomic loci using a double-stranded DNA cytidine deaminase. (FIG. 1B) Aggregate profile plot showing distribution of cytidine deamination fractions (y axis) around CTCF binding sites (left) and transcription start sites (TSS, right) from whole-genome sequencing (WGS) of DddA11-treated K562 cells. (FIG. 1C) TDAC-seq and chromatin tracks at the DSCR4/8 locus across a 9.4 kb region in K562 and GM12878 cells, showing cell-type-specific chromatin accessibility. TDAC-seq signal represents the average number of DddA11 mutations in a 50-bp window. C-to-T edits and G-to-A edits are depicted. (FIG. 1D) TDAC-seq tracks showing the footprint of dCas9 protein after introducing dCas9.sgRNA in MOLM-13 cells. C·G-to-T·A edits are also depicted. (FIG. 1E) Top: genome tracks showing chromatin profiles, TDAC-seq, and DADs identified for each single DNA molecule at the DSCR4/8 locus. Bottom: heatmap showing DAD co-accessibility, where the gray indicates the observed co-accessibility fraction minus the expected value, calculated as the product of individual accessibility fractions. (FIG. 1F) Box plot showing the fraction of reads covered by a DAD that overlaps a given ATAC peak in each of 15 TDAC-seq datasets, stratified by the ATAC signal strength. Box plots show the median and interquartile range (IQR), with whiskers extending to the minimum and maximum values. (FIG. 1G) Box plots showing the distribution of DAD lengths across 951,754 reads in 15 TDAC-seq datasets, grouped according to overlap with ATAC peaks. There are 129,284 DADs in the “False” group and 282,067 DADs in the “True” group. Box plots show the median and IQR, with whiskers extending to the minimum and maximum values, excluding outliers that are beyond 1.5× IQR. Welch's two-sided t-test was used to calculate p-value (p<1.0×10⁻⁴). Data in FIGS. 1B-1G are representative of n=2 replicates.

FIGS. 2A-2F. TDAC-seq measures CRISPR perturbation and chromatin structure from the same single DNA molecule. (FIG. 2A) Genome tracks showing ATAC-seq, TDAC-seq, and footprint scores at the indicated CTCF binding site in MOLM-13 cells. TDAC-seq signal represents the average number of DddA11 edits in a 50-bp window. The footprint window radii are 73 and 25 bp for nucleosome and TF footprints, respectively. (FIG. 2B) Schematic of how TDAC-seq detects both CRISPR deletions and chromatin accessibility simultaneously from the same single DNA molecule, allowing partitioning of reads with and without CRISPR deletions. (FIG. 2C) Genome tracks showing TDAC-seq and footprint scores from DNA reads containing CRISPR deletions near the sgRNA binding site, which overlaps the CTCF and YY1 motifs shown in FIG. 2A. Magnified inset (top) highlights the CRISPR deletions. (FIG. 2D) Four sgRNA sequences targeting CTCF and/or YY1 motifs from UniBind at the HOXA locus. (FIG. 2E) Box plot of the number of DddA11 edits in the accessible region (bar in FIG. 2A) from reads with and without CRISPR deletions after CRISPR perturbation with individual sgRNAs. Boxes represent the IQR, horizontal lines indicate the median, and whiskers extend to 1.5× IQR. Outliers are shown as individual dots. (FIG. 2F) Swarm plot showing the ratio of DddA11 edits in CRISPR-deleted reads versus wild-type (WT) reads at the HOXA locus across different motif deletion categories. ‘Δ’ indicates that the TF motif is partially or fully deleted, while ‘-’ indicates the TF motif remains undisturbed. The genotype with the YY1 motif deleted while the CTCF motif remained intact was not detected. Data from two replicates of CRISPR perturbation using four individual sgRNAs (HOXA_sgRNA-1,2,3, and 4) are combined. Each dot represents a single genotype. Data in FIGS. 2A, 2C, and 2E are representative of n=2 replicates.

FIGS. 3A-3F. TDAC-seq detects chromatin accessibility dynamics at the β-globin locus in erythroid-differentiated HSPCs. (FIG. 3A) ATAC-seq tracks showing the β-globin locus at indicated stages of CD34+ HSPC erythroid differentiation. (FIG. 3B) Genome tracks showing chromatin accessibility changes measured by TDAC-seq at indicated stages of CD34⁺ HSPC erythroid differentiation at the HBD-HBB locus. TDAC-seq signal represents the average number of DddA11 edits in a 50-bp window. (FIG. 3C) Schematic of TDAC-seq combined with CRISPR-Cas9 targeting HBG1/2 promoters in CD34⁺ HSPCs. Cas9·sgRNA-68 targets two sites in the HBG1/2 locus, which after cutting predominantly results in a hybrid fusion product containing an ˜5 kb deletion. TDAC-seq was conducted five days after inducing erythroid differentiation. (FIG. 3D) Flow cytometry histograms of HbF levels (PE, x axis) in erythroid-differentiated HSPCs (day 5) treated with Cas9 and a non-targeting sgRNA (top) or sgRNA-68 (bottom). (FIG. 3E) ATAC-seq from erythroid-differentiated HSPCs (day 5) treated with Cas9 and a non-targeting sgRNA or sgRNA-68. Although sgRNA-68 produces a hybrid fusion, ATAC-seq reads were aligned to both the HBG1 and HBG2 promoters due to homologous DNA sequences (bars). (FIG. 3F) TDAC-seq of the three most abundant genotypes with different deletion sizes at the HBG locus from erythroid-differentiated HSPCs (day 5) transduced with sgRNA-68, compared with non-targeting sgRNA control. TDAC-seq signals were aggregated from the top 10% of highly DddA11-mutated reads. The dotted line indicates the sgRNA-68 cut sites. Data in FIG. 3B are representative examples of n=2 replicates. Data in FIG. 3E are mean of n=2 replicates, where ATAC-seq was independently conducted on the same cells. Data FIG. 3F presents n=2 replicates, where TDAC-seq was independently conducted on cells electroporated separately.

FIGS. 4A-4E. TDAC-seq can measure the impact of pooled CRISPR perturbations on chromatin accessibility in parallel. (FIG. 4A) Schematic of TDAC-seq integrated with pooled CRISPR-mutational scanning to measure the effects of CRISPR perturbations on chromatin accessibility at targeted CREs. (FIG. 4B) Top: Wild-type K562 ATAC-seq and a line plot showing the fraction of reads (y axis) with CRISPR deletions across the HS2 region (x axis). Bottom: TDAC-seq heatmaps in K562 cells following CRISPR-Cas9 cutting at HS2. Reads were grouped by 541 CRISPR deletion genotypes (y axis), with black bars indicating deletions. TDAC-seq signal represents the average number of DddA11 edits in a 100-bp window, with wild-type signal subtracted across the HS2 region (x axis). (FIG. 4C) Most abundant genotypes detected near the sgRNA-10 and sgRNA-11 cut sites (left), and corresponding DddA11 mutation rates (%), calculated as the number of DddA11 edits divided by the total number of editable sites within the accessible region (bar in FIG. 4B). Box plots show the median, IQR, and full range excluding outliers beyond >1.5× IQR. Means are indicated by triangles. sgRNA cutting sites and selected transcription factor binding sites from UniBind are shown above the sequences. Genotypes were clustered by the 10-bp deletion window (shaded box). Deletions overlapping the key motif sequence (underline) show the greatest reduction in accessibility. (FIG. 4D) Genome tracks showing ATAC-seq signal (y axis) after individual transduction of a non-targeting sgRNA, sgRNA-1, and sgRNA-11 into K562 cells. The dotted lines indicate the sgRNA cut sites. (FIG. 4E) Bar plot showing mRNA levels of HBE, HBG1/2, and HBB after individual transduction of sgRNA1 and sgRNA11 as measured by RT-qPCR. Expression is normalized to non-targeting sgRNA. Bars represent mean±s.d. of n=6 replicates. Compared to the non-targeting sgRNA, sgRNA-11 significantly reduced gene expression, with p=9×10⁻¹², p<1.0×10⁻¹⁰, and p=1.9×10⁻³ for the respective genes (two-tailed Student's t-test). Data in FIGS. 4B-4C are representative examples of n=2 replicates. Data in FIG. 4D are mean of n=2 replicates, where ATAC-seq was independently conducted on the same cells.

FIGS. 5A-5C. TDAC-seq can measure the impact of pooled ABE perturbations on chromatin accessibility across distinct genotypes. (FIG. 5A) Top: wild-type K562 ATAC-seq and a line plot showing the ratio of A·T-to-G·C edit coverage in ABE (+) versus ABE (−) samples across the HS2 region. Bottom: heat maps of TDAC-seq using ABE on the HS2 enhancer in K562 cells. Individual sequencing reads were grouped by the positions of A·T-to-G·C base edits, with black lines indicating the edited locations on the HS2 locus. TDAC-seq signal represents the average number of DddA11 edits in a 100-bp window. For each genotype, TDAC-seq signal subtracted by wild-type signal are shown across the HS2 region. Genotypes were grouped by the number of ABE edits. (FIG. 5B) Volcano plot showing the number of DddA11 edits in the accessible region (bar in FIG. 5A) and −log₁₀P for each genotype (Welch's two-sided t-test). Wild-type reads have about 30 DddA11 edits in average (dotted line). Each dot (genotype) indicates the number of ABE edits. Top hits are labeled by the positions of ABE edits. (FIG. 5C) Box plots (right) showing the number of DddA11 edits for reads with the indicated genotype (left). Genotypes are clustered by whether the labeled A·T base pair is mutated, and only genotypes with ≥1000 reads are shown. sgRNA binding sites and selected transcription factor binding sites from UniBind are shown above the sequences. The core motifs of FOXK2 (top) and FOS/JUN (AP-1) (bottom) are underlined. DddA11 edits are counted over the accessible region (bar in FIG. 5A). Box plots show the median and IQR, with whiskers extending to the minimum and maximum values, excluding outliers that are beyond 1.5× IQR. Mean is indicated by triangles. Data in FIGS. 5A-5C are representative examples of n=2 replicates.

FIGS. 6A-6C. TDAC-seq detects different CRISPR perturbations changing GFI1B enhancer accessibility in CD34+ HSPCs. (FIG. 6A) Genome tracks showing ATAC-seq and TDAC-seq signals at the GF11B enhancer in K562 cells. TDAC-seq was performed using either DddA11 or MGYFPDa829. C·G-to-T·A edits are depicted. (FIG. 6B) Top: Genome tracks showing ATAC-seq from unperturbed CD34+ HSPCs, the fraction of reads containing a CRISPR deletion at the indicated position, and TDAC-seq signals from wild-type reads across the GF11B enhancer region (x axis). TDAC-seq signal represents the average number of MGYFPDa829 edits in a 100-bp window. Bottom: Heat maps showing the difference in MGYFPDa829 edits within the accessible region (chr9: 133003719-133004894) for each genotype compared to wild-type reads. Each dot represents a distinct genotype, where the x-axis indicates the CRISPR deletion start site and the y-axis indicates deletion length. The each dot indicates the change in the number of MGYFPDa829 edits within the accessible region between CRISPR-edited reads and wild-type reads, where edits within the CRISPR deletion are ignored. Dot size reflects statistical significance (p-value calculated by Welch's two-sided t-test). Shaded regions mark key motifs whose deletion reduces accessibility, as identified through motif clustering. (FIG. 6C) Box plots of the difference in MGYFPDa829 edits within the accessible region (chr9: 133003719-133004894) for genotypes containing full or partial deletions of the sequences highlighted in shaded boxes, compared to wild-type reads. “Other” includes all remaining CRISPR deletion genotypes. TF motifs from UniBind56,57 are labeled above the sequence. Box plots show the median and IQR, with whiskers extending to the minimum and maximum values, excluding outliers that are beyond 1.5×IQR. Each genotype is represented by a black dot, except in the “Other” category where only outliers are shown as unfilled dots. Data in FIGS. 6A-6C are representative examples of n=2 replicates.

FIGS. 7A-7D. MGYFPDa829 enhances sensitivity for detecting chromatin accessibility. (FIG. 7A) Genome tracks at HS2 locus showing ATAC-seq and two TDAC-seq replicates under different conditions, including changes in deaminase concentration and reaction time, addition of UGI, and substitution of DddA11 with MGYFPDa829. TDAC-seq signal represents the average number of DddA11 or MGYFPDa829 edits in a 100-bp window. (FIG. 7B) CTCF binding sites (left) and transcription start sites (TSS, right) from WGS in K562 cells treated with MGYFPDa829. (FIG. 7C) TDAC-seq using MGYFPDa829 and chromatin tracks at the DSCR4/8 locus (left) and ZBTB38 locus (right) in K562 cells. TDAC-seq signal represents the average number of MGYFPDa829 edits in a 100-bp window. C·G-to-T·A edits are depicted. (FIG. 7D) Genome tracks showing chromatin accessibility changes measured by TDAC-seq using MGYFPDa829 at indicated stages of CD34+ HSPC erythroid differentiation at the HBD-HBB locus. Data in FIGS. 7B-7D are representative examples of n=2 replicates.

### DETAILED DESCRIPTION

Cis-regulatory elements (CREs) are DNA sequences that function as switches to control the expression of genes. CREs often densely cluster in linear sequence space and contain finer-scale elements that together control transcription factor (TF) binding, chromatin accessibility, and downstream expression of associated genes. Methods that profile chromatin accessibility have provided high-resolution views of the function and actuation of CREs. Alternatively, CRISPR genome-editing technologies have enabled the systematic perturbation and fine mapping of endogenous CREs. For instance, high-resolution CRISPR mutational scanning of the BCL11A enhancer and HBG1/2 promoter regions followed by readout of fetal hemoglobin (HbF) levels has provided key insights into specific elements and TF motifs that control HbF expression, ultimately culminating in breakthrough therapies to treat Sickle Cell Disease. Understanding how genome editing affects chromatin accessibility is essential for elucidating the molecular mechanisms that link genetic alterations to downstream phenotypic outcomes.

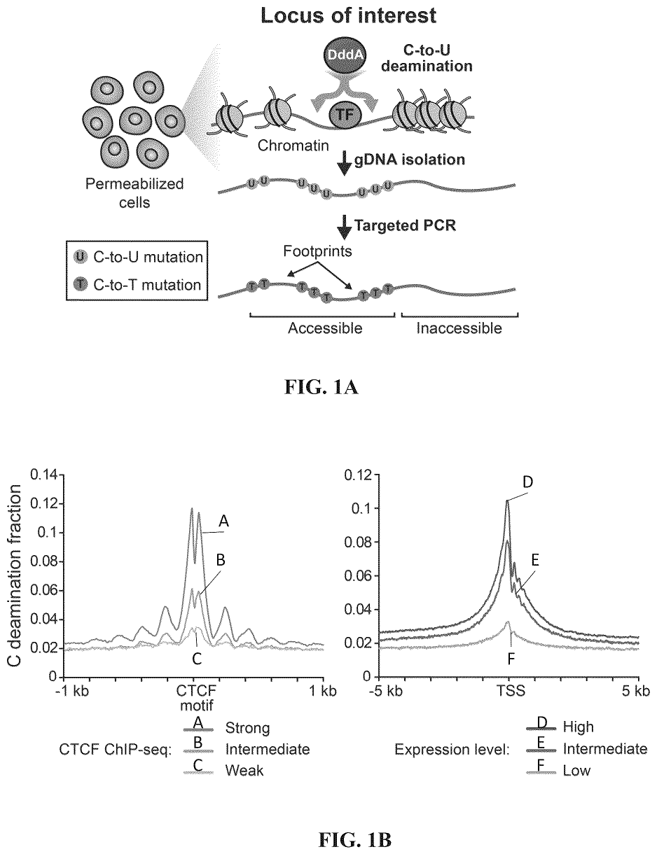

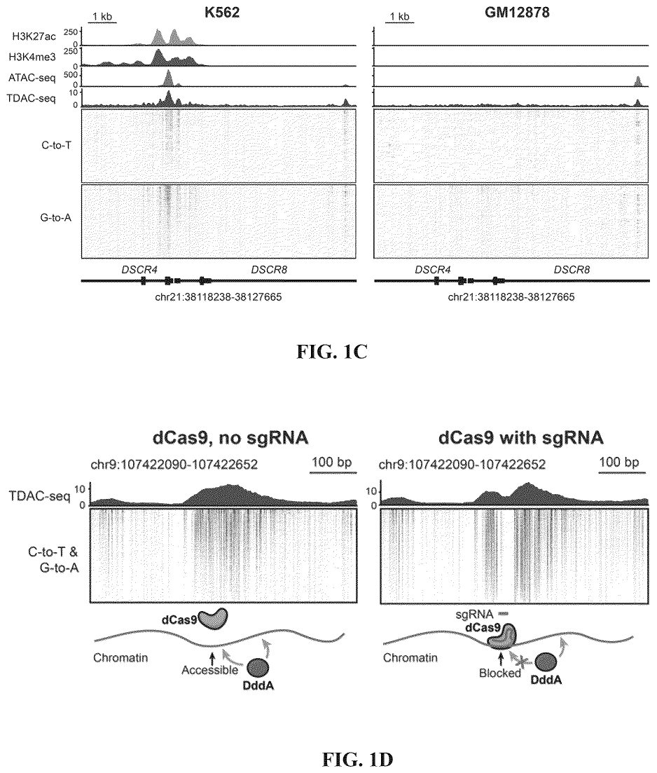

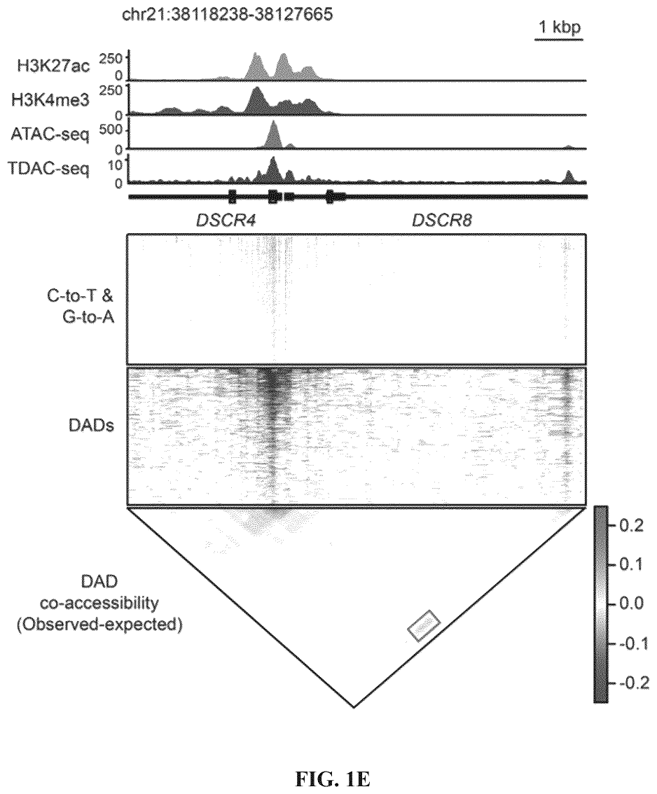

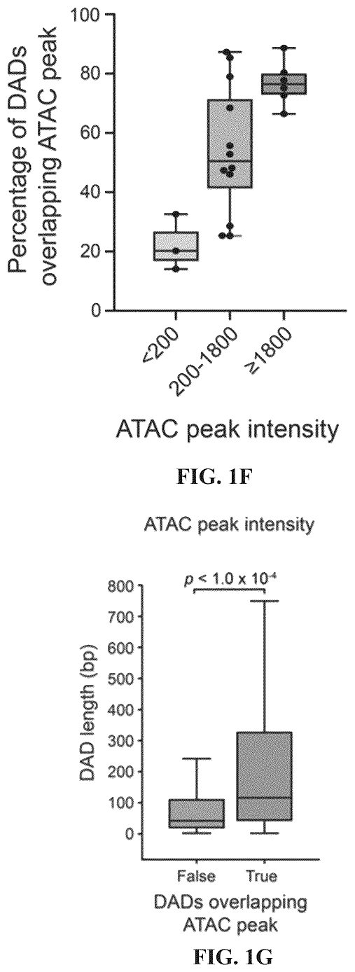

Technologies to jointly map and perturb CREs are needed and will drive basic understanding of DNA regulatory logic and development of gene therapies. Whereas traditional short-read measurements rely on fragmenting bulk populations of chromatin fibers, long-read sequencing formats retain information about crosstalk and interactions across a single chromatin fiber and enables synchronous readout of genetic variants and their effect on chromatin accessibility. However, these long read-compatible methods require higher DNA input amounts because they typically rely on methyltransferases to ‘stencil’ nucleosome and TF positioning, which are detected by sequencing the unnatural, methylated bases directly and are thus challenging to combine with PCR-based amplification. These limitations restrict their ability to study specific high-value loci at high coverage (>1000×), preventing integration with pooled CRISPR-Cas9 or base editor mutational scanning to measure the functional effects of genetic variants at scale. To address these limitations, DddA, a double-stranded DNA cytidine deaminase, was leveraged to profile chromatin accessibility of targeted genomic loci using long-read sequencing after PCR amplification (FIG. 1A). This method is called Targeted Deaminase-Accessible Chromatin sequencing (TDAC-seq). TDAC-seq can be leveraged with CRISPR perturbations to comprehensively read out editing outcomes with single-nucleotide resolution, enabling us to simultaneously map their direct impact on chromatin accessibility for hundreds of unique genotypes in a massively parallel pooled format.

### Methods

Aspects of the present disclosure relate to a method of identifying therapeutic genomic targets within a genome. A therapeutic genomic target can be any target of interest, including, without limitation, genes of interest and/or gene regulatory elements of interest. In some embodiments, a therapeutic genomic target is a locus in a gene exon. In some embodiments, a therapeutic genomic target is a locus in a gene intron. In some embodiments, a therapeutic genomic target is a locus in a gene. In some embodiments, a therapeutic genomic target is a locus in a gene promoter. In some embodiments, a therapeutic genomic target is a locus in a gene enhancer. In some embodiments, a therapeutic genomic target is a locus in a BCL11A enhancer. In some embodiments, a therapeutic genomic target is associated with genetic variants have been linked to myeloproliferative neoplasm risk. In some embodiments, a therapeutic genomic target is associated with GWAS (Genome-Wide Association Study) variants. In some embodiments, a therapeutic genomic target is an HBG1 promoter. In some embodiments, a therapeutic genomic target is an HBG2 promoter. In some embodiments, the therapeutic genomic target is associated with a blood disorder. In some embodiments, the therapeutic genomic target is associated with anemia. In some embodiments, the therapeutic genomic target is associated with sickle cell anemia. In some embodiments, the therapeutic genomic target is associated with beta-thalassemia.

Genomic mutations are implicated in a wide range of clinical indications, including monogenic inherited disorders such as cystic fibrosis (CFTR mutations), sickle cell disease (HBB mutations), Duchenne muscular dystrophy (DMD mutations), and Tay-Sachs disease (HEXA mutations). Mutations also underlie many cancer indications, where somatic alterations in genes such as TP53, KRAS, BRAF, EGFR, and PIK3CA drive tumor initiation, progression, and therapeutic response across malignancies including breast, colorectal, lung, melanoma, and hematologic cancers. In the realm of hematologic and immunologic diseases, genomic variants contribute to conditions such as β-thalassemia (HBB mutations), severe combined immunodeficiency (ADA or IL2RG mutations), and chronic granulomatous disease (CYBB mutations). Numerous neurologic and neurodevelopmental indications are also rooted in genomic abnormalities, including Huntington's disease (HTT expansions), amyotrophic lateral sclerosis (SOD1, C9orf72), Rett syndrome (MECP2), and various epileptic encephalopathies (SCN1A, STXBP1). In metabolic and endocrine disorders, mutations in genes such as PAH (phenylketonuria), GAA (Pompe disease), GBA (Gaucher disease), and MEFV (familial Mediterranean fever) lead to characteristic systemic phenotypes. Additionally, cardiovascular indications such as hypertrophic cardiomyopathy (MYH7, MYBPC3), long QT syndrome (KCNQ1, KCNH2), and familial hypercholesterolemia (LDLR, APOB, PCSK9) arise from pathogenic variants affecting cardiac structure, electrical signaling, or lipid metabolism. Further, genomic mutations contribute to a broad spectrum of rare multisystem syndromes, including Marfan syndrome (FBN1), Noonan syndrome (PTPN11), and mitochondrial disorders resulting from mutations in nuclear or mitochondrial genes. A therapeutic genomic target can be any of the non-limiting exemplary targets.

Accordingly, some aspects of the technology relate to a method for identifying a therapeutic genomic target sequence, the method comprising: (a) perturbing a genomic locus using a gene editing system; (b) deaminating one or more nucleotides of the genomic locus using a deaminase to produce a pattern of deamination at the genomic locus; and (c) identifying a therapeutic genomic target sequence based on the pattern of deamination at the genomic locus.

“Perturbation” herein includes any one or more modification to a genomic locus, including but not limited to, nucleic acid deletions, insertions, substitutions and combinations thereof (e.g., “indels,” i.e., insertion and deletion).

A “gene editing system” includes one or more molecule that enables precise modifications to a genome, which can be used to modify (e.g., insert, delete, and/or substitute specific nucleic acid sequence(s) within a genome). Non-limiting examples of gene editing systems include CRISPR-Cas systems, TALEN (Transcription Activator-Like Effector Nuclease) systems, ZFN (Zinc Finger Nuclease) systems, and meganuclease (homing endonuclease) systems. In some embodiments, the gene editing system is a CRISPR-Cas system. In some embodiments, the CRISPR-Cas system comprises a guide RNA (gRNA) and a Cas nuclease, for example, selected from Cas9 enzymes and Cas12 enzymes. In some embodiments, a guide RNA comprises the nucleotide sequence of any one of SEQ ID NOs: 1-10.

In some embodiments, the CRISPR-Cas system comprises a deactivated Cas (dCas) nuclease. A “deactivated Cas” or “dCas” can include a modified form of a Cas nuclease (e.g., Cas9 or Cas12) that lacks (e.g., has been engineered to lack) DNA-cutting activity while retaining its DNA-binding activity guided by a single-guide RNA (sgRNA).

In some embodiments, a CRISPR-Cas system further comprises a base editor, for example, selected from cytosine base editors and adenine base editors. A “base editor” enables direct, irreversible conversion of one DNA base into another without introducing double-stranded breaks or requiring a donor DNA template. Base editors include two main components: a deactivated Cas or a Cas nickase and a base-editing enzyme (i.e., an enzyme that catalyzes the conversion of one base to another). Non-limiting examples of base-editing enzymes include deaminases such as cytosine base editors and adenine base editors. In some embodiments, a cytosine base editor is used to mark sites within a chromosome. In some embodiments, a cytosine base editor is used to edit sites within a chromosome. In some embodiments, an adenine base editor is used to mark sites within a chromosome. In some embodiments, an adenine base editor is used to edit sites within a chromosome.

In some embodiments, a CRISPR-Cas system comprises a Cas nickase (nCas). A “Cas nickase” includes a modified version of a Cas nuclease (e.g., Cas9 or Cas12) that can introduce a single-stranded DNA break (or “nick”) rather than a double-stranded break. This can be achieved by inactivating one of the two nuclease domains of a Cas nuclease.

In some embodiments, a CRISPR-Cas system further comprises a reverse transcriptase (i.e., an enzyme that synthesizes complementary DNA (cDNA) from an RNA template.)

In some embodiments, a deaminase is a double-stranded DNA deaminase. A “double-stranded DNA deaminase” can include an enzyme that catalyzes the chemical modification of cytosine (C) or adenine (A) bases in both strands of double-stranded DNA (dsDNA), leading to the conversion of these bases into others. Non-limiting examples of a double-stranded DNA deaminase include double-stranded DNA deaminase toxin A (DddA), DddB, DddSs, DddDd-5, MGYFPDa829, and CseDa01. In some embodiments, a DddA is DddA11. DddA11 is an engineered variant of the bacterial cytidine deaminase enzyme DddA (double-stranded DNA deaminase toxin A) from Burkholderia cenocepacia.

In some embodiments, a genomic locus comprises a cis-regulatory element. A cis-regulatory element can include a short sequence of DNA near or within a gene that regulates the expression of that gene. Non-limiting examples of cis-regulatory elements include promoters, enhancers, silencers, insulators, and response elements (e.g., hormone response elements and heat shock elements).

In some embodiments, a method comprises amplifying a genomic locus. Gene amplification refers to the process of increasing the number of copies of a specific gene. In some embodiments, a genomic locus is amplified using polymerase chain reaction (PCR) to produce amplicons of the genomic locus. In some embodiments, the method comprises half-nested PCR with a long extension time (15 minutes) using KOD One PCR Master Mix to increase the PCR product yield.

In some embodiments, a method further comprises sequencing the amplicons of the genomic locus. In some embodiments, sequencing of the amplicons comprises long-read sequencing, for example, nanopore sequencing or single-molecule real-time sequencing.

Other aspects of the technology relate to a method for screening for guide RNAs targeting regulatory elements that modulate chromatin accessibility, the method comprising: (a) perturbing genomic loci using a CRISPR-Cas system comprising a pooled library of guide RNAs; (b) deaminating one or more nucleotides of the respective genomic loci using a deaminase to produce a pattern of deamination at the respective genomic loci; and (c) identifying guide RNAs targeting regulatory elements that modulate chromatin accessibility based on the pattern of deamination at the respective genomic loci.

“Chromatin assembly” refers to the degree to which DNA within chromatin is available for interactions with proteins, such as transcription factors, polymerases, and other regulatory molecules. Accessibility is largely determined, for example, by the structure and organization of chromatin, which can exist in a compact (heterochromatin) or relaxed (euchromatin) state.

A “pooled library of guide RNAs” can include a collection of (e.g., thousands to hundreds of thousands of) distinct gRNAs designed to target specific DNA or RNA sequences across a genome or transcriptome. A pooled gRNA library can include several components. Each guide RNA (gRNA) in the library can contain a spacer sequence, for example, 20 nucleotides long, that is complementary to a specific DNA or RNA target sequence. These gRNAs can be paired with Cas enzymes, such as Cas9, for DNA cleavage, for example. In some embodiments, a pooled library includes a diverse collection of gRNAs targeting genes, regulatory elements, and/or specific loci of interest. To deliver the library into cells, for example, the gRNAs can be cloned into vectors, such as lentiviruses or plasmids, for transduction.

Deamination includes the enzymatic removal of an amine group, converting cytosine to uracil (U) or adenine to hypoxanthine (which base pairs like guanine). A “pattern of deamination” can include, for example, a specific sequence and positional preferences of cytosine (C) or adenine (A) base deamination within a defined region of the genome.

In some embodiments, the method of the present disclosure further comprises adding a uracil-DNA glycosylase inhibitor (UGI). A UGI is a molecule that inhibits the activity of endogenous uracil-DNA glycosylase (UDG). UDG is a DNA glycosylase that removes deaminated cytosine from DNA. Accordingly, the addition of UGI to the method of the present disclosure increases the efficiency and activity of the deaminases of the present disclosure.

Aspects of the present disclosure related to a method of identifying a therapeutic genomic target, tiling the therapeutic genomic target with guide RNAs, and identifying guide RNAs that confer a desired effect. In some embodiments, a desired effect is gene activation. In some embodiments, a desired effect is gene inactivation. In some embodiments, a guide RNA targets a gene exon. In some embodiments, a guide RNA targets a gene intron. In some embodiments, a guide RNA targets a gene enhancer. In some embodiments, a guide RNA targets a gene promoter. In some embodiments, gene editing is used to determine the effect of deleting a region of a gene on chromatin remodeling, inferred from the pattern of deamination. In some embodiments, these conclusions are drawn by aligning sequencing reads to the reference genome, optionally using Minimap2, grouping all reads by their genotype, and then averaging the deamination rate across reads in each group. In some embodiments, the average is taken in a sliding window of N base pairs, where N is optionally 50. In some embodiments, the reads in each group are downsampled to the same read count per group. In some embodiments, genotypes are excluded from analysis if the gene edit is unlikely to be generated from a guide RNA in the library. In some embodiments, where adenine base editor was used, this is optionally determined as a gene edit that does not occur in positions +3 to +9 of the guide RNA. In some embodiments, where CRISPR nuclease was used, this is optionally determined as a genomic deletion smaller than 5 base pairs. As a skilled artisan will appreciate, sequencing reads will contain PCR duplicates, which may bias the interpretation of results. In some embodiments, the stochastic pattern of deamination is used as a unique molecular identifier (UMI). A UMI is a sequence of nucleotides at specific positions of a DNA sequence that uniquely identifies that DNA molecule and would be propagated after PCR, enabling sequencing reads with the same UMI to be deduplicated. In some embodiments, deduplication is performed by removing all except one sequencing read for each UMI.

As described herein, the methods of the present disclosure use a deaminase to identify therapeutic genomic targets. Roh H. et al. Nature Methods, 22, 2083-2093 (2025), including all additional information, extended data, supplementary information, source data, and code (github.com/liaulab/TDAC-seq), is incorporated herein in its entirety. As a skilled artisan will appreciate, deaminase editing is biased when DddA11 is used but not when MGYFPDa829 is used. “Editing bias” includes a phenomenon in which certain mutations are more likely to occur than others due to deaminase sequence bias. To correct this sequence bias, deproteinized DNA was treated with the deaminase and the editing rate on this naked DNA corresponds to and enables the correction of the enzyme's intrinsic sequence bias. In addition, computational methods are used for the identification of long stretches of mutated bases corresponding to continuous open chromatin regions, referred to as deaminase-accessible DNA sequences (DADs). To correct variable density of deaminase-editable bases in different loci, DADs are called, in some embodiments, using a model. In some embodiments, a model is a hidden Markov model (HMM) with emission probabilities corresponding to the intrinsic sequence bias of the deaminase. In some embodiments, these values are empirically derived from naked DNA as described above. In some embodiments, these values are indirectly derived by fitting the HMM to TDAC-seq data. The term “Markov model,” as used herein, includes a stochastic model in probability theory used to model pseudo-randomly changing systems. A Markov model assumes that future states depend only on the current state, not on the events that occurred before it (that is, it assumes the Markov property). A hidden Markov model (HMM) is a Markov model in which the observations are dependent on a latent (or hidden) Markov process (referred to as X). An HMM requires that there be an observable process Y whose outcomes depend on the outcomes of X in a known way. Since X cannot be observed directly, the goal is to learn about the state of X by observing Y. In some embodiments, Y corresponds to the deamination state of a nucleotide in a DNA sequence while X corresponds to whether that nucleotide is in a DAD region.

In some embodiments, footprints were computed either on individual DNA molecules or aggregated reads. In some embodiments, for each single base-pair position of interest. the number of deamination events were computed for a center region with a given radius r, as well as two flanking regions with a diameter of r. Similarly, the sum of deaminase sequence bias was computed for the same center and flanking regions. Then the depletion of deamination events at the center was calculated using a left-tailed center-versus-flank binomial test using bias_center/(bias_center+bias_flank) as the probability p of binomial distribution. A pseudo-count was added to prevent division of zero. Testing was performed for the left and right flank separately, and the least significant p-value was kept as the final p-value. Eventually, the −log₁₀ (p-value) was used as the footprint score.

### Kits

The components described herein may, in some embodiments, be assembled into kits to facilitate their use in research applications. Non-limiting examples of components described herein include deaminase, gene editing systems, and uracil-DNA glycosylase inhibitors (UGIs). In some embodiments, a kit of the present disclosure comprises a deaminase and a gene editing system. In some embodiments, a kit of the present disclosure comprises a deaminase and a UGI. In some embodiments, a kit of the present disclosure comprises a deaminase, a gene editing system, and a UGI.

In some embodiments, a deaminase is a double-stranded DNA deaminase. In some embodiments, a double-stranded DNA deaminase is double-stranded DNA deaminase toxin A (DddA), DddA11, DddB, DddSs, DddDd-5, MGYFPDa829, or CseDa01.

In some embodiments, a DddA is DddA11. In some embodiments, a gene editing system is a CRISPR-Cas system, a TALEN (Transcription Activator-Like Effector Nuclease) system, a ZFN (Zinc Finger Nuclease) system, or a meganuclease (homing endonuclease) system. In some embodiments, a CRISPR-Cas system comprises a guide RNA (gRNA) and a Cas nuclease. In some embodiments, a Cas nuclease is a Cas9 enzyme. In some embodiments, a Cas nuclease is a Cas12 enzyme. In some embodiments, a Cas nuclease is a deactivated Cas (dCas) nuclease. In some embodiments, a Cas nuclease is a Cas nickase (nCas). In some embodiments, a CRISPR-Cas system further comprises a base editor. In some embodiments, a base editor is a cytosine base editor. In some embodiments, a base editor is an adenine base editor. In some embodiments, a CRISPR-Cas system further comprises a reverse transcriptase.

A kit may include one or more containers housing the components of the disclosure and instructions for use. Specifically, such kits may include one or more agents described herein, along with instructions describing the intended application and the proper use of these agents. Kits for research purposes may contain the components in appropriate concentrations or quantities for performing various experiments.

The kit may be designed to facilitate use of the methods described herein by researchers and can take many different forms. Each of the compositions of the kit, where applicable, may be provided in liquid form (e.g., in solution), or in solid form (e.g., a dry powder). In certain cases, some of the compositions may be constitutable or otherwise processable (e.g., to an active form), for example, by the addition of a suitable solvent or other medium (for example, water or a cell culture medium), which may or may not be provided in the kit. As used herein, “instructions” can include a component of instruction and/or promotion, and typically involve written instructions on or associated with the packaging. Instructions also can include any oral or electronic instructions provided in any manner such that a user will clearly recognize that the instructions are to be associated with the kit, for example, audiovisual (e.g., videotape, DVD, CD-ROM, website links for downloadable file), internet, and/or web-based communications, etc. The written instructions may be in a form prescribed by a governmental agency regulating the manufacture, use, or sale of biological products, which instructions can also reflect approval by the agency of manufacture, use, or sale for plant administration.

The kit may contain any one or more of the components described herein in one or more containers. As an example, in one embodiment, the kit may include instructions for mixing one or more components of the kit and/or isolating and mixing a sample and applying it to a plant. The kit may include a container housing the compounds described herein. The compounds may be in the form of a liquid, gel, or solid (powder). The compounds may be prepared sterilely, packaged in a syringe, and shipped refrigerated. Alternatively, the compounds may be housed in a vial or other container for storage. A second container may have other agents prepared sterilely. Alternatively, the kit may include the compounds premixed and shipped in a syringe, vial, tube, or other container.

### EXAMPLES

### Example 1. TDAC-Seq for Mutational Profiling of Chromatin Accessibility on Long, Targeted Loci

We sought to develop a method for employing DddA to mutationally profile chromatin accessible regions on long DNA molecules via C to U conversion, which preserves DNA integrity and creates C·G-to-T·A mutations after PCR amplification (FIG. 1A). For this purpose, we employed DddA11, an evolved variant of the deaminase DddA that possesses relaxed sequence bias. Briefly, cell nuclei were isolated, permeabilized, and treated with purified DddA11. We first performed whole-genome sequencing of DddA11-treated K562 cells using Oxford Nanopore Technologies to assess C·G-to-T·A mutation rates and their positions. Consistent with prior reports, DddA11 preferentially deaminates cytidine preceded by a 5′-thymidine or 5′-cytidine (data not shown). Across the whole genome, C·G-to-T·A mutations were enriched at DNase I hypersensitive sites, CTCF sites, and active promoters, indicating that DddA11 preferentially deaminates accessible chromatin with a periodic mutation distribution consistent with strong nucleosome phasing at these regions (FIG. 1B). Altogether, these results demonstrate the ability of DddA11 to mark accessible chromatin by mutational profiling.

Since chromatin accessibility profiling with DddA11 does not cleave the DNA molecule but superimposes a mutational signature, our method is compatible with targeted PCR amplification and long-read sequencing technologies, allowing us to enrich specific genomic loci of interest with high coverage. To this end, we PCR-amplified fifteen genomic loci of interest after DddA11 treatment and sequenced the amplicons (˜3 to 11.5 kb in length) by nanopore sequencing. We then deduplicated reads using DddA11-induced mutations as unique molecular identifiers, assuming there is a very low likelihood of observing kilobase-sized molecules with identical distributions of stochastic C·G-to-T·A mutations (see Methods). This workflow results in TDAC-seq signal at the targeted loci of interest at high coverage, which were concordant with ATAC-seq data (FIG. 1C). Furthermore, dynamic changes to chromatin accessibility caused by dCas9 binding could be robustly detected by TDAC-seq (FIG. 1D), highlighting the sensitivity of our approach and ability to detect protein occupancy that occludes chromatin accessibility.

Because TDAC-seq provides single-molecule readouts of chromatin accessibility across long DNA reads, we next investigated whether it could reveal correlations between accessible sites on single chromatin fibers that are otherwise obscured in bulk population average measurements (e.g., ATAC-seq). We examined the patterns of DddA11-induced mutations across individual DNA molecules and, as in previous approaches, observed long stretches of mutated bases corresponding to continuous open chromatin regions, which we refer to as deaminase-accessible DNA sequences (DADs) (FIG. 1E). To correct for the variable density of DddA11-editable bases in different loci, we called DADs using a hidden Markov model with emission probabilities corresponding to the intrinsic sequence bias of the enzyme (data not shown). As validation of the DAD calling approach, across all sequencing reads in the fifteen loci of interest, a median of 90.4% of DADs overlapped with ATAC-seq peaks, and DADs within ATAC-seq peaks were significantly longer than those outside (FIGS. 1F,G). Next, we calculated per-read correlation of TDAC-seq signal across the respective loci. Consistent with prior reports, our analysis revealed nearby distal TDAC-seq accessible sites were correlated and open together across single chromatin fibers, highlighting the strength of long-read sequencing in detecting co-occurring accessibility (FIG. 1E). Altogether, these results establish TDAC-seq as a robust method to mutationally profile chromatin accessibility at target genomic loci of interest at high coverage.

### Example 2. TDAC-Seq Reveals Chromatin Dynamics Induced by CRISPR Perturbations

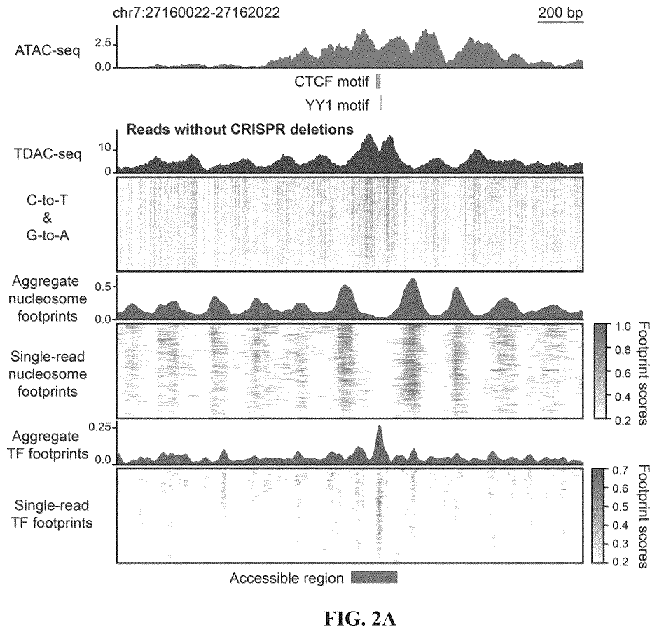

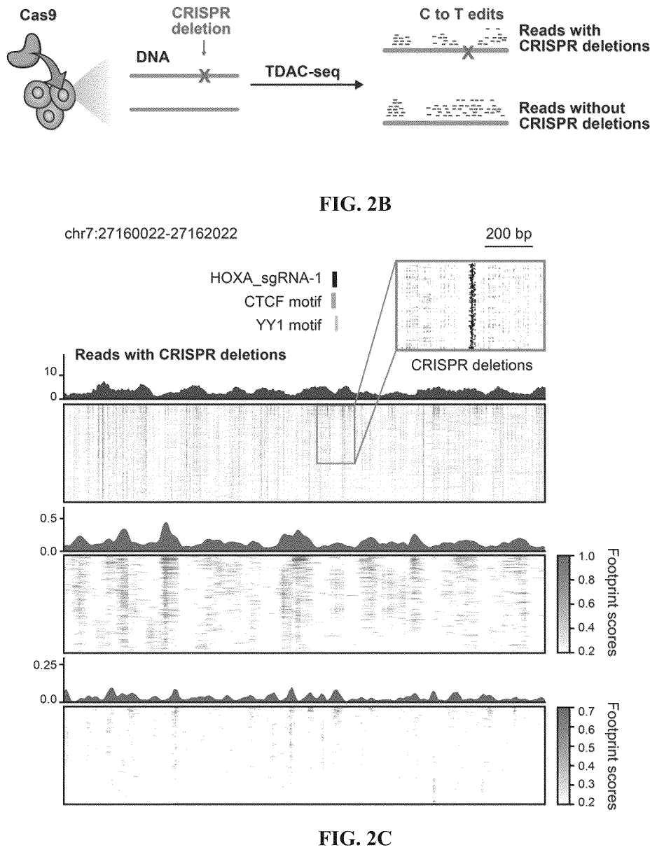

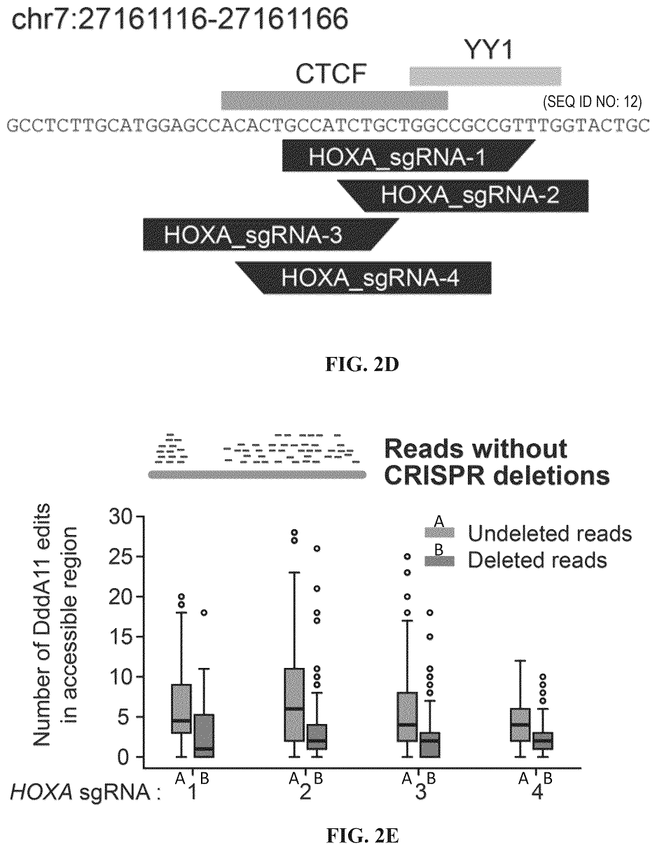

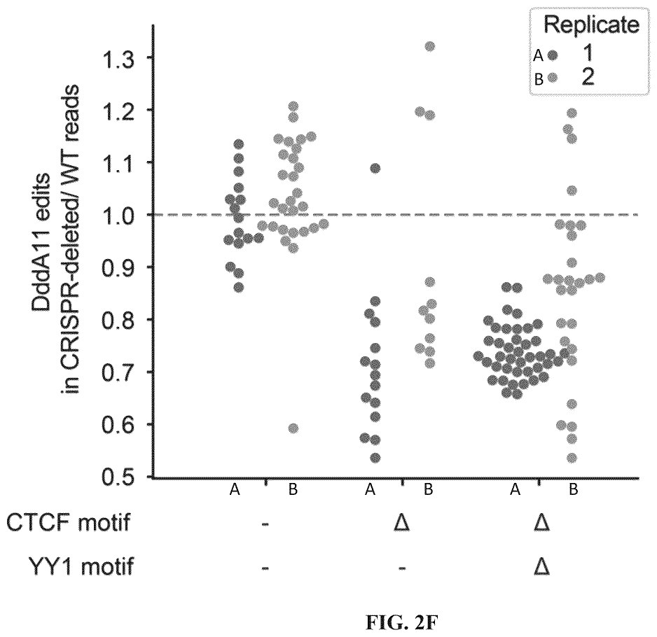

In addition to measuring chromatin accessibility, we further tested the ability of DddA11 mutations to footprint nucleosomes and TFs, where the depletion of deamination-induced mutations within accessible chromatin could indicate protein binding. We calculated such depletion in varying footprint window sizes (1-99 bp) to detect footprints of objects with diverse sizes, where TFs were detected using smaller windows and nucleosomes are detected with larger windows (data not shown). To ensure that mutation rates reflect chromatin accessibility, the intrinsic sequence bias of DddA11 was measured and normalized using a bias model generated from deproteinized, naked gDNA treated with DddA11. Whole-genome sequencing of DddA11-treated nuclei revealed that different TFs display distinct patterns and intensities of footprints and associated nucleosome positioning. The aggregate TF footprint scores correlate with DNase-seq footprint scores, particularly for TFs known to exhibit strong footprinting in DNase-seq and ATAC-seq (data not shown). Nucleosome footprints from TDAC-seq align well with previously obtained MNase-seq data (data not shown). In addition, TDAC-seq on a region containing a CTCF binding site revealed the presence of strong CTCF footprints across single molecules (FIG. 2A). Altogether, TDAC-seq leverages DddA11 mutations to detect both nucleosome positioning and binding of TFs that produce strong footprints, providing single-read resolution within specific genomic loci that are PCR amplified.

Next, we tested whether TDAC-seq could distinguish changes in chromatin accessibility and footprinting following CRISPR perturbation. CRISPR-Cas9-mediated disruption of a CTCF binding motif in the HOXA locus was previously demonstrated to disrupt CTCF binding and decrease chromatin accessibility within the region. A sgRNA targeting this position and Cas9 were delivered into MOLM-13 cells, which was followed by TDAC-seq at this region. We then deconvoluted CRISPR-edited versus non-edited reads as well as the superimposed DddA11 mutational signature simultaneously by nanopore sequencing (FIG. 2B). Compared to non-edited reads, DNA strands where the CTCF motif was disrupted showed reduced chromatin accessibility as well as weaker nucleosome and TF footprints (FIGS. 2A,C). Moreover, since TDAC-seq affords single-nucleotide resolution, we could scrutinize the CRISPR deletions to determine the minimal deletion necessary to reduce chromatin accessibility (FIG. 2D). This analysis revealed that deletions spanning the CTCF and YY1 motifs displayed the largest differential TDAC-seq signal, consistent with the previously reported roles of CTCF and YY1 in regulating chromatin accessibility. Together, these results demonstrate how TDAC-seq can simultaneously detect both CRISPR perturbations and chromatin accessibility from a single DNA molecule at single-nucleotide resolution.

### Example 3. High-Resolution Profiling of Chromatin Accessibility in the β-Globin Locus During Erythroid Differentiation of CD34+ HSPCs

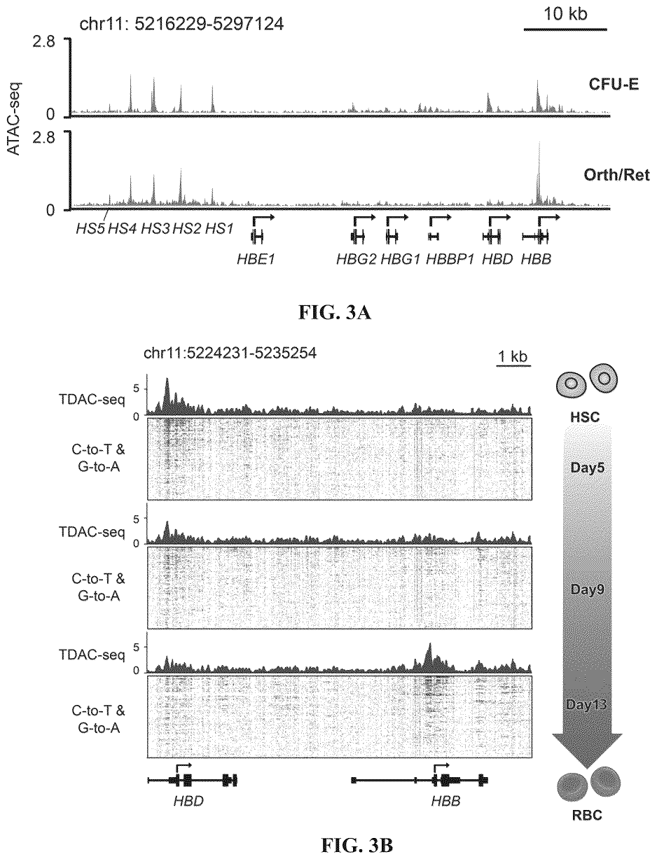

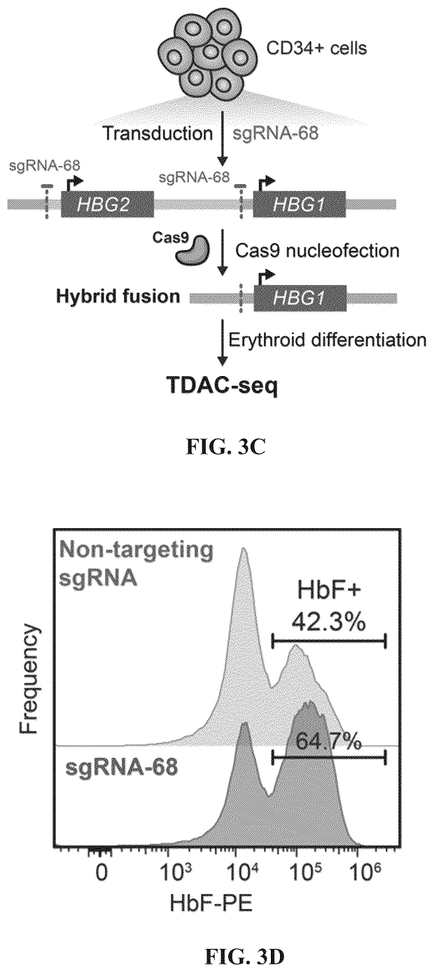

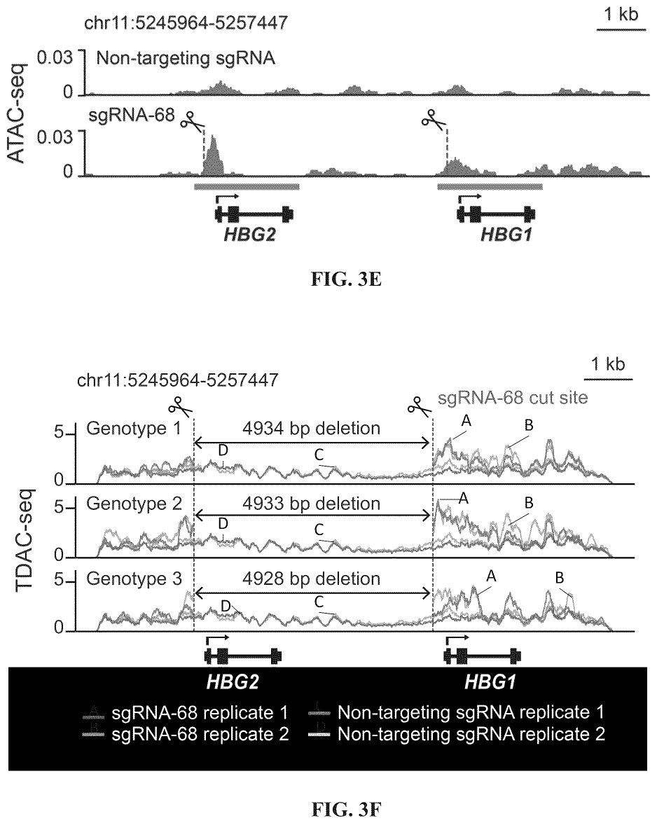

Profiling chromatin accessibility of targeted loci of interest at high coverage from primary cells remains challenging due to limiting amounts of input gDNA, rendering it difficult to achieve sufficient sequencing coverage. Since DddA-induced mutations are uniquely compatible with targeted PCR amplification, we posited that TDAC-seq could enable the high-resolution readout of chromatin accessibility for CREs of interest in primary cells. Consequently, we tested if TDAC-seq could resolve changes in chromatin accessibility in the β-globin locus upon erythroid differentiation of CD34+ hematopoietic stem cell progenitor cells (HSPCs) (FIG. 3A). During erythroid differentiation, HBB and HBD gain and lose chromatin accessibility, respectively, and TDAC-seq of an 11 kb amplicon spanning the promoters of these two genes could robustly detect these expected changes (FIG. 3B).

Notably, the β-globin locus also encompasses the paralogous genes HBG1 and HBG2, which control the expression of γ-globin during development, and have attracted interest for their therapeutic relevance but have been challenging to disentangle. Although typically silenced in adult blood, reactivation of γ-globin by mutations associated with hereditary persistence of HbF reduces the severity of Sickle Cell Disease. Due to this connection, the promoter regions of HBG1 and HBG2 have been targeted by CRISPR-based therapeutics in patient-derived CD34+ HSPCs to induce reactivation of γ-globin and HbF expression for the treatment of Sickle Cell Disease. This includes OTQ923, an autologous in ex vivo CRISPR-Cas9-edited CD34+ product, which ultimately progressed into patients. Intriguingly, sgRNA-68, the sgRNA component of OTQ923, targets the promoters of both HBG1 and HBG2 due to their high levels of sequence homology. Subsequent DNA repair and ligation of double-strand DNA break ends lead to a ˜5 kb deletion that excises HBG2 entirely and creates a single hybrid gene with part of the HBG2 promoter sequence fused to HBG1 (FIG. 3C). However, the impact of CRISPR edits on the locus structure and chromatin accessibility has been difficult to precisely measure by short-read-based approaches (ATAC-seq) because HBG1 and HBG2 are contiguous, duplicated genes spanning ˜7 kb, preventing the alignment of short reads to either specific gene region due to their high levels of sequence homology.

To determine whether our long-read approach could address these limitations, we conducted TDAC-seq on the HBG1-HBG2 locus in CD34+ HSPC-derived erythroblasts, which were transduced with either a non-targeting sgRNA-control or sgRNA-68, electroporated with Cas9 protein, and induced to differentiate for 5 days (data not shown)—sufficient time to allow complete Cas9 turnover. We verified that Cas9 sgRNA-68 led to the creation of a large ˜5 kb deletion as the major product (data not shown), which upon differentiation resulted in robust upregulation of HbF (data not shown). Due to the high sequence homology between the two promoters, short sequencing reads obtained from bulk ATAC-seq cannot distinguish them or detect these large deletions from the mixture of products resulting from Cas9 treatment (FIG. 3E). By contrast, the large deletions could be clearly called for each TDAC-seq read, where the 4928-bp, 4933-bp, and 4934-bp deletions accounted for the vast majority of CRISPR mutational outcomes (data not shown). Notably, chromatin accessibility could be measured simultaneously on reads containing these deletions, which displayed similarly elevated TDAC-seq signals in the promoter of the hybrid gene locus (FIG. 3F), consistent with strong upregulation of HbF induced by sgRNA-68 and the notion that the deleted region may contain repressive elements. Altogether TDAC-seq enables a high-resolution view of the effects of OTQ923 per genotype on the γ-globin locus in primary CD34+ HSPCs upon differentiation.

### Example 4. Integrating TDAC-Seq with Pooled CRISPR-Mutational Scanning

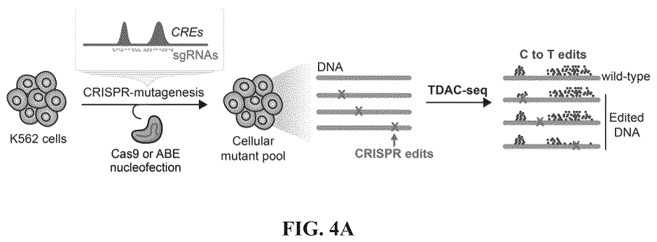

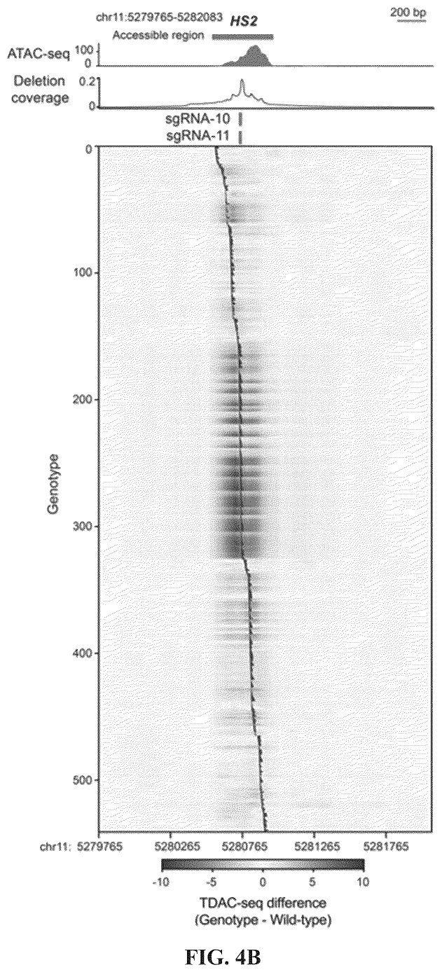

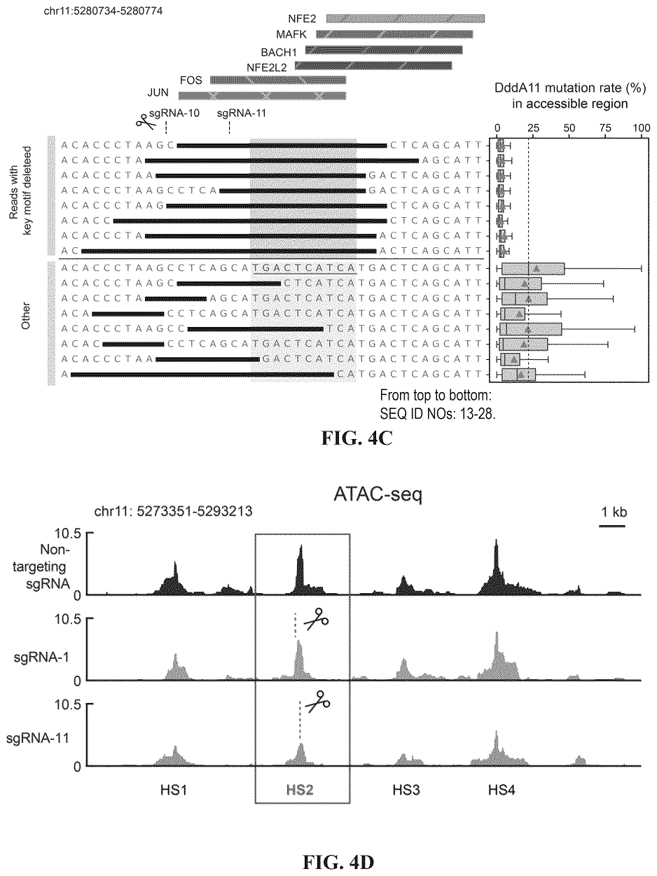

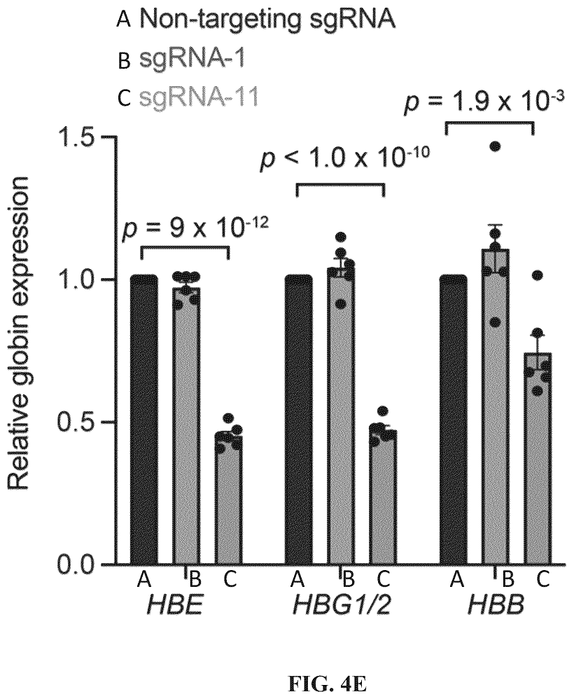

Given the high coverage afforded by TDAC-seq at targeted genomic loci of interest and the ability of our protocol to simultaneously detect CRISPR deletions and chromatin accessibility, we considered whether TDAC-seq could be combined with pooled CRISPR-mutational scanning to systematically dissect CREs to identify elements that control chromatin accessibility (FIG. 4A). A pooled library consisting of 21 sgRNAs tiling hypersensitive site 2 (HS2) of the globin locus control region was transduced into K562 cells followed by electroporation of Cas9 and TDAC-seq after 6 days. Due to the stochasticity of DNA repair of the double-stranded breaks created by Cas9, we observed 541 unique CRISPR-induced deletion genotypes with sufficient sequencing coverage (>400 unique reads) to simultaneously measure their impact on accessibility (data not shown). We quantified C·G-to-T·A aggregate mutation rates per each genotype across the amplicon (FIG. 4B). DNA molecules bearing CRISPR deletions had variable TDAC-seq signal depending on the position and size of the CRISPR deletions. Notably, deletions attributed to sgRNA-10 and sgRNA-11 reside close to the center of the HS2 ATAC-seq peak and resulted in significantly reduced TDAC-seq signal. We clustered reads with these deletions to identify a shared minimal sequence whose deletion is necessary and sufficient for this effect (data not shown; see Methods). This analysis revealed a 10-bp sequence that corresponds to the AP-1 (JUN/FOS) motif, which has previously been demonstrated to be essential for activity of HS2. Reads where this motif is entirely deleted had significantly reduced DddA accessibility at HS2 compared to those that partially delete the motif or that do not perturb it (FIG. 4C). Although CRISPR deletions will remove a small number of C·G base pairs that could have been edited by DddA, the number of DddA edits within these deletions is negligible relative to this effect size (data not shown). We also confirmed that individual introduction of sgRNA-11, as opposed to sgRNA-1 or a non-targeting control sgRNA, resulted in decreased ATAC-seq signal at HS2 as well as decreased expression of proximal globin genes (FIGS. 4D-4E). While bulk ATAC-seq provides a readout of the aggregate effects arising from a mixture of editing outcomes (data not shown), TDAC-seq can precisely deconvolute the effects of individual editing outcomes on chromatin accessibility.

### Example 5. TDAC-Seq Enables Detection of Cooperative ABE Effects on CRE Accessibility

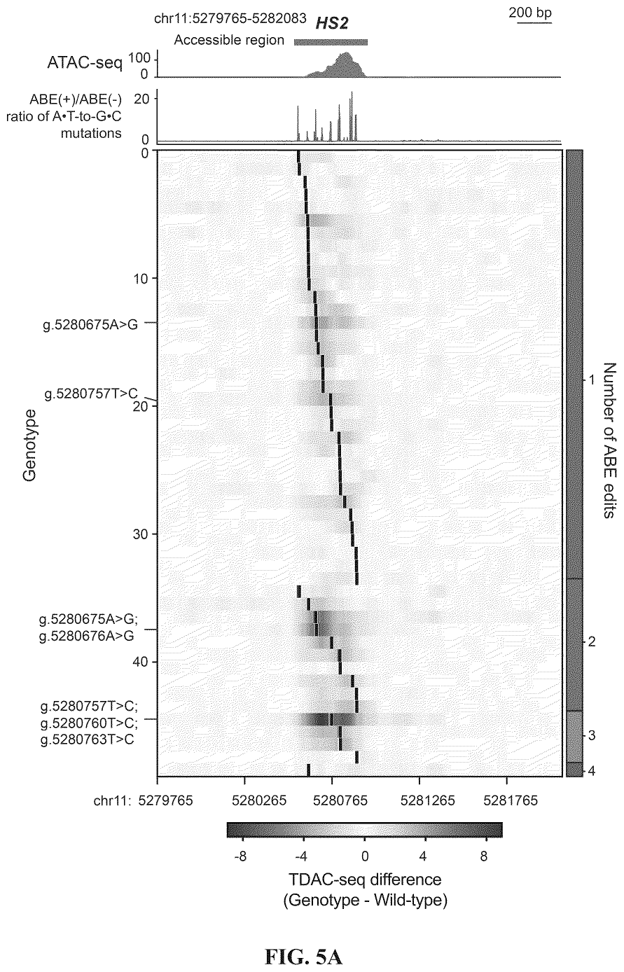

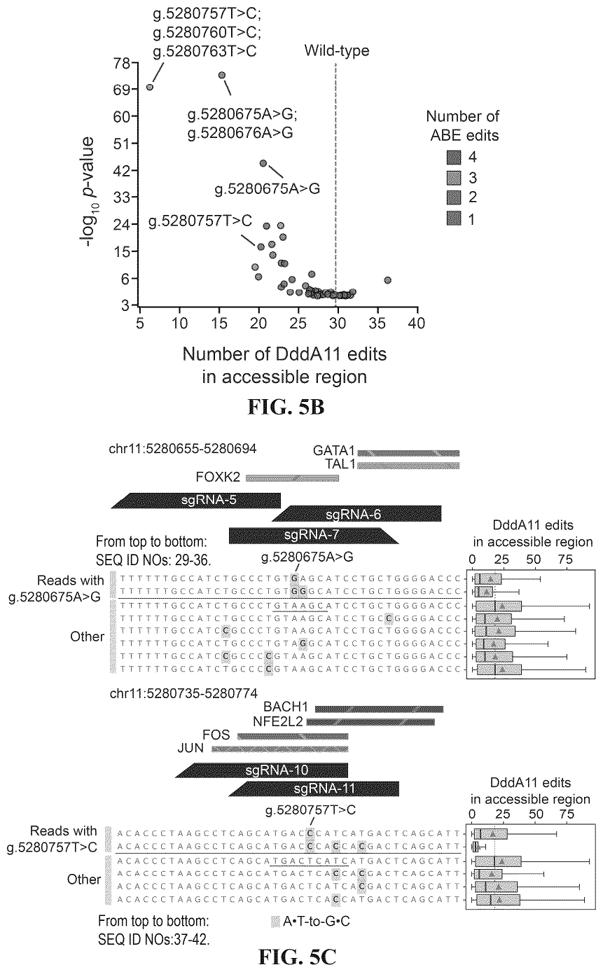

Next, we sought to test the compatibility of TDAC-seq with finer-scale genetic perturbations afforded by base editors, specifically the adenosine base editor 8e (ABE8e) that yields A·T-to-G·C mutations. Purified ABE8e was electroporated into K562 cells transduced with the same HS2-tiling sgRNA library as before and TDAC-seq was then performed (FIG. 4A). A·T-to-G·C mutations were strongly enriched at the predicted editing windows and target strands of the sgRNAs, consistent with editing by ABE (FIG. 5A: upper panel). Because many sgRNAs contain more than one adenosine within the ABE editing window, many reads stochastically had more than one A·T-to-G·C mutation. As a result, we observed 49 unique genotypes created by ABE with sufficient sequencing coverage (>100 unique reads) (data not shown). To evaluate the impact of ABE edits on TDAC-seq signal, we grouped reads by the position of ABE edits, computed the average TDAC-seq signal of each genotype, and compared with wild-type reads (FIG. 5A, lower panel). The impact of these single base edits on TDAC-seq signal was attenuated in comparison to deletions afforded by Cas9 nuclease, consistent with the generally weaker effect size of point mutations. Like with the deletions produced by Cas9 nuclease, ABE mutations targeting the center of HS2 exhibited the most significant reductions in TDAC-seq signal.

Because TDAC-seq can directly genotype base edits as opposed to indirectly inferring from sgRNA deconvolution, we considered whether the effects of multiple simultaneous base edits-which often occur when there are multiple adenosines within a sgRNA's editing window-could be interrogated. TDAC-seq reads containing multiple edits (representing 18.6% of all sequencing reads) could be robustly detected, and as anticipated, tend to show stronger reduction in HS2 accessibility compared to reads with single base edits, highlighting cooperative effects (FIG. 5B). Notably, while the g.5280675A>G mutation in the FOXK2 motif reduces HS2 accessibility and had the strongest effect among reads with a single base edit-consistent with the reported cooperativity between FOXK2 and AP-1-combining it with g.5280676A>G yielded even stronger effects (FIG. 5C, upper panel). Likewise, the g.5280757T>C mutation in the AP-1 motif was the second strongest individual base edit in our pool and combining that mutation with the g.5280760T>C and/or g.5280763T>C mutations reduced accessibility even more than any of these mutations individually (FIG. 5C, lower panel). Altogether, these findings show that TDAC-seq can be integrated with pooled CRISPR-mutational scanning at scale to dissect sequence-function relationships governing enhancer accessibility at single-nucleotide resolution.

### Example 6. TDAC-Seq with MGYFPDa829 Enables Large-Scale Mutational Scanning in CD34⁺ HSPCs

Next, we scaled up our CRISPR-Cas9 scanning approach to include more than 100 sgRNAs and applied it to primary CD34⁺ HSPCs. We targeted an enhancer downstream of the GF11B promoter where genetic variants have been linked to myeloproliferative neoplasm risk. Prior studies have shown that deletion of this entire 1.6 kb enhancer reduces GF11B expression. However, the exact identity and position of functional sites within this enhancer region remained unknown. To dissect sequence-function relationships at this locus, we employed TDAC-seq in combination with high-density CRISPR-Cas9 scanning in CD34⁺ HSPCs.

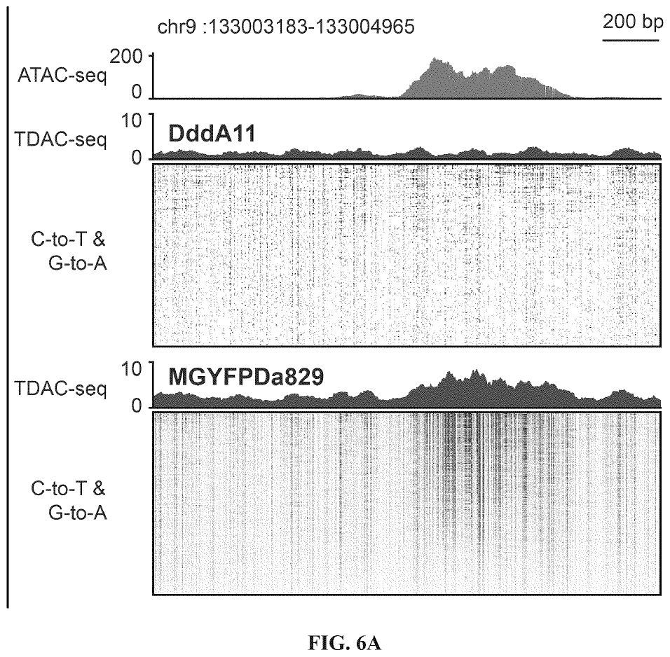

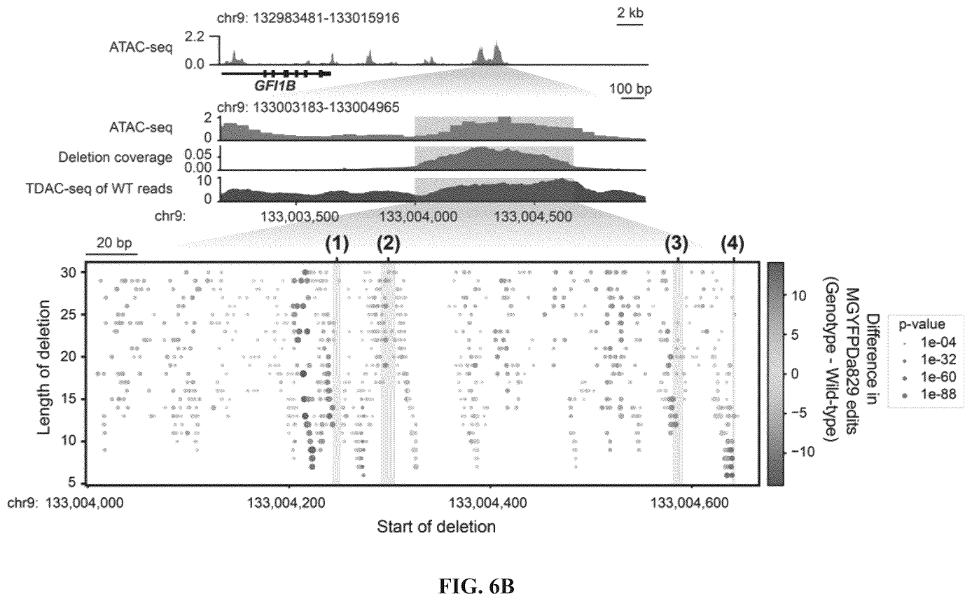

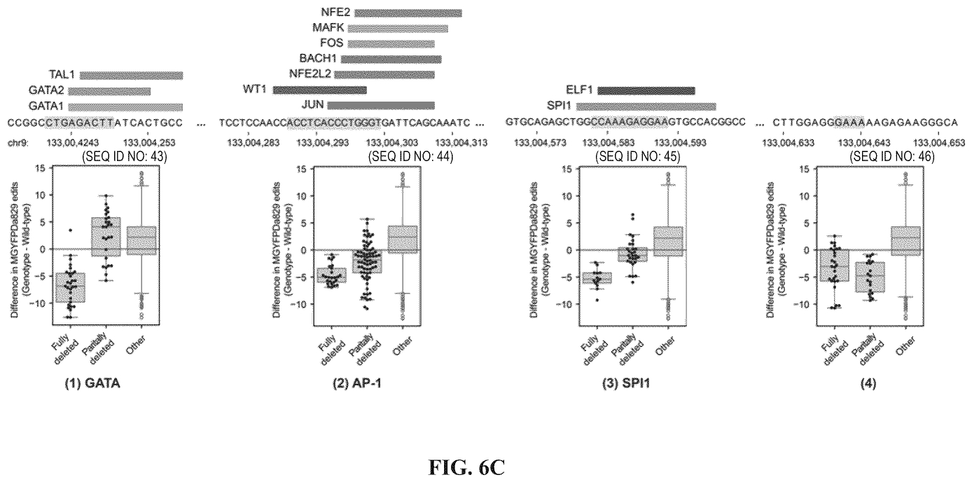

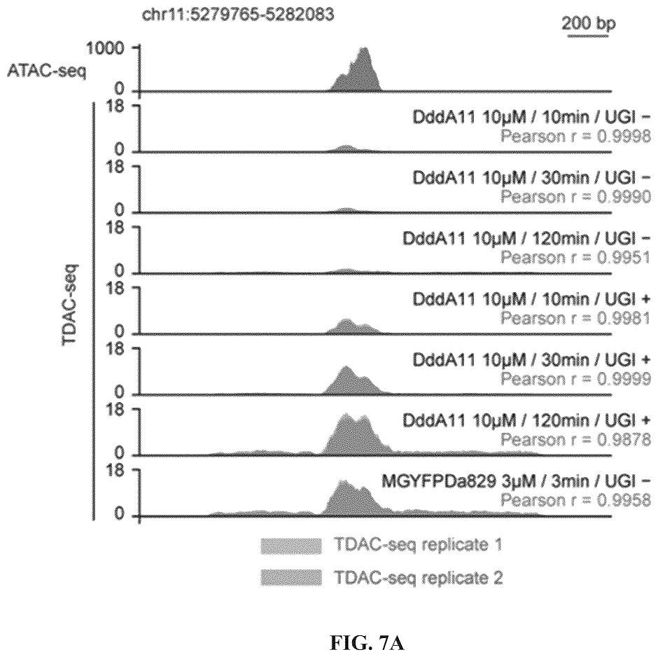

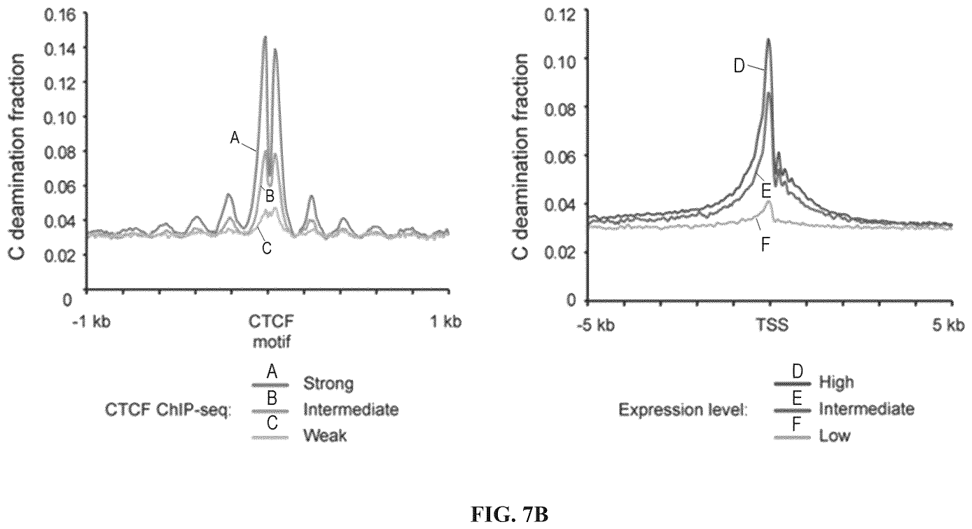

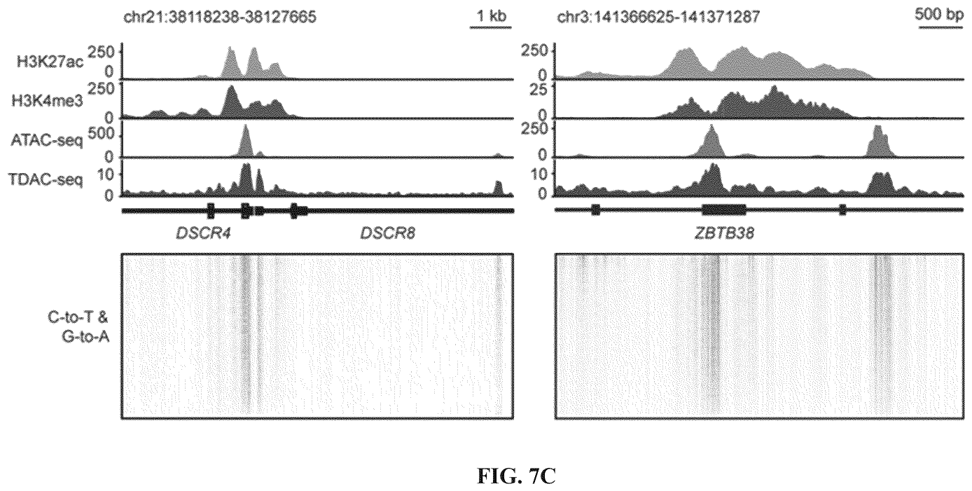

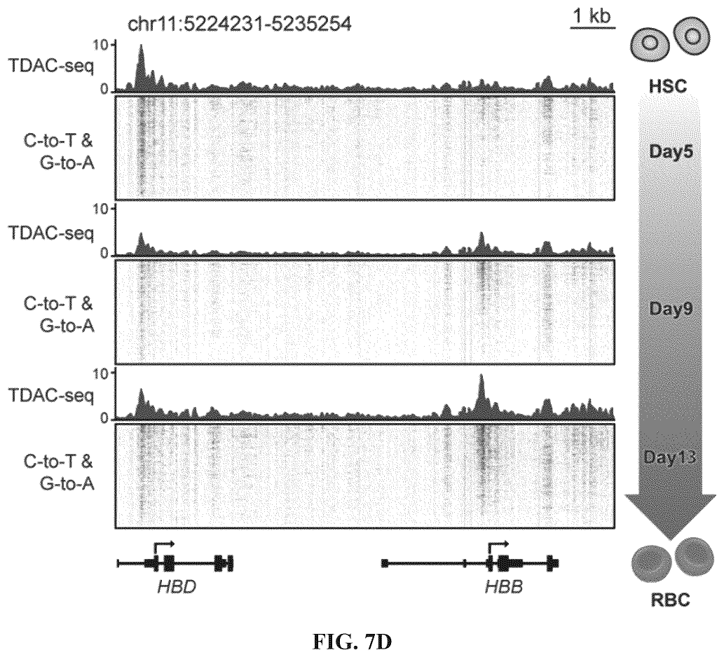

While TDAC-seq performed well in detecting highly open regions, its sensitivity declined in more closed regions such as the GF11B enhancer, due to the lower editing efficiency of DddA11 (FIG. 6A, upper panel). To address this limitation, we found that adding uracil-DNA glycosylase inhibitor (UGI) enhanced the DddA11 mutation rate by inhibiting endogenous uracil-DNA glycosylase, which would otherwise excise DddA11-induced mutations (FIG. 7A). To further improve editing efficiency, we purified and tested a more active double-stranded cytidine deaminase, MGYFPDa829, that was recently identified from a systematic bioinformatics survey. This enzyme lacks sequence context preference, thereby improving resolution and reducing sequence bias. MGYFPDa829 yielded similar results in both WGS and TDAC-seq compared to DddA11 (FIGS. 7B-7D). Notably, a 3-minute reaction with MGYFPDa829 achieved a mutation rate comparable to a 2-hour DddA11 reaction, substantially increasing TDAC-seq sensitivity, especially in weakly accessible regions (FIG. 7A). When applied to the GF11B enhancer, MGYFPDa829-based TDAC-seq improved detection of accessibility compared to DddA11 (FIG. 6A).

To gain a high-resolution functional map of this GF11B enhancer, we performed MGYFPDa829-based TDAC-seq coupled with CRISPR-Cas9 scanning of this GF11B enhancer in CD34⁺ HSPCs using a library of 138 sgRNAs (data not shown). This generated 947 distinct CRISPR deletion genotypes with over 3,000-fold coverage per genotype at this locus (data not shown). Notably, the scale of data achieved in this study is uniquely enabled by targeted locus PCR in TDAC-seq. Compared to Cas9-based targeted locus enrichment, TDAC-seq achieved 187- to 683-fold increase in coverage in the target regions across samples tested (data not shown). This high coverage represents a key advantage of TDAC-seq that enables fine-scale mapping in primary cells with limited cell numbers.

Deletions of varying lengths and positions within the GF11B enhancer differentially modulated chromatin accessibility of this region (data not shown). We quantified TDAC-seq signal for each genotype normalized to that in wild-type reads and clustered genotypes to reveal minimal motifs whose deletion altered chromatin accessibility (FIGS. 6B-6C). This motif clustering analysis revealed that deletion of GATA, AP-1, and SPI1 motifs led to the most significant decrease in accessibility, consistent with the essential role of these TFs in hematopoiesis. Altogether, these results showcase the capabilities of TDAC-seq to enable the large-scale functional dissection of key enhancer elements in primary cells and to reveal unique biological insights not accessible by other methods.

In summary, TDAC-seq combined with CRISPR-mutational scanning provides targeted and scalable methods to fine-map the activity and sequence determinants of DNA cis-regulatory elements in high resolution across hundreds of editing outcomes. This integration is uniquely possible by leveraging a double-stranded DNA cytidine deaminase that mutationally profiles chromatin fibers in a manner compatible with PCR of the targeted loci, enabling the sequencing coverage and resolution at the locus of interest necessary for conducting a pooled CRISPR screen. Importantly, CRISPR edits can lead to diverse outcomes and potential combinatorial effects, and these details are not fully distinguished by only capturing sgRNA identity. Notably, TDAC-seq can precisely characterize heterogeneous editing outcomes directly while simultaneously profiling associated chromatin states, allowing us to dissect finer TF motifs controlling the accessibility of CREs at single-nucleotide resolution. More broadly, we anticipate that TDAC-seq will provide a high-throughput platform to systematically evaluate the impact of sgRNAs for CRISPR-Cas9 and base editing therapeutic strategies for the treatment of human genetic diseases.

### Methods Related to Examples 1-6

DddA11 expression and purification

DddA11 was cloned into pETDuet-1::dddAₜₒₓ-dddI_{A} by introducing six mutations in the DddA protein coding sequence (S1330I, A1341V, N1342S, E1370K, T1380I, T1413I). Mutant sequences were generated by PCR and introduced using NEBuilder HiFi DNA Assembly Master Mix (NEB E2621L). After obtaining the pETDuet-1::dddA11ₜₒₓ-dddI_{A} plasmid, a TEV cleavage site and an 8-amino-acid linker sequence, ‘GGGSGGGS (SEQ ID NO: 11),’ followed by the MBP tag, were inserted in-frame after the protein coding sequence.

The plasmid was transformed into BL21 (DE3)pLysS E. coli cells. After plating, a single colony was cultivated in LB media with 100 mg/L ampicillin at 37° C. for 6 h. This culture was used to inoculate 12 L of LB broth at a 1:100 dilution, and the culture was grown at 37° C. until it reached an OD600 of 0.6. Expression was induced with 0.5 mM isopropyl β-D-thiogalactoside (IPTG), and the culture was incubated at 18° C. for 16 h. Cells were harvested by centrifugation at 4000 g for 35 min, and each 3 L pellet was resuspended in 40 mL binding buffer (5 mM imidazole, 0.5 M NaCl, and 20 mM Tris-HCl, pH 7.9) containing 1 mg/mL lysozyme. The cells were sonicated at 60% amplitude for 10 min (10 s on, 10 s off) on ice, and the lysate was centrifuged at 25,000×g for 40 min.

All protein purification steps were performed at 4° C. A column was loaded with His60 nickel resin (Takara Bio), washed with 5 column volumes (cv) of Milli-Q water, and then with 6 cv of binding buffer. The supernatant was added to the charged resin and incubated on a rotator for 20 min. The supernatant was allowed to flow through the column, followed by sequential washes with 20 cv of binding buffer, 10 cv of wash buffer I (60 mM imidazole, 0.5 M NaCl, 20 mM Tris-HCl, pH 7.9), 5 cv of wash buffer II (100 mM imidazole, 0.5 M NaCl, 20 mM Tris-HCl, pH 7.9), and 7 cv of elution buffer (500 mM imidazole, 0.5 M NaCl, 20 mM Tris-HCl, pH 7.9, 1 mM DTT). Eluates were directly analyzed by Coomassie-stained SDS-PAGE. Fractions containing DddA11-MBP-DddI were collected and subjected to buffer exchange using a concentrator (Amicon Ultra-15 Centrifugal Filter Unit, 30 kDa MWCO) in refolding wash buffer (5 mM imidazole, 50 mM Tris-HCl pH 7.5, 500 mM NaCl, and 1 mM DTT). The concentrated protein was added to 350 mL of denaturing buffer (8 M urea, 5 mM imidazole, 50 mM Tris-HCl pH 7.5, 500 mM NaCl, and 1 mM DTT) and incubated for 36 h at 4° C. with shaking. A fresh Ni resin was washed with 50 cv of Milli-Q water and 10 cv of 8 M urea denaturing buffer. The urea buffer containing the eluted proteins was loaded onto a gravity-flow column, incubated for 20 min, and the flow-through was collected. Sequential washes were performed with 7 cv of denaturing buffer with decreasing urea concentrations (8 M, 6 M, 4 M, 2 M, 1 M), followed by a final wash with refolding wash buffer to remove residual urea. Proteins bound to the resin were eluted with 3 cv of wash buffer 1, followed by 1 cv of wash buffer 2, and finally 5 cv of elution buffer. Protein-containing fractions were identified by SDS-PAGE and subsequently subjected to two rounds of buffer exchange using a 30 kDa concentrator in loading buffer (20 mM Tris pH 8, 20 mM NaCl, 1 mM DTT). The concentrated samples were purified by anion exchange chromatography using fast protein liquid chromatography (FPLC) with a 5 mL HiTrap Q HP column (Cytiva). The column was equilibrated in low-salt buffer (20 mM Tris-HCl pH 8, 1 mM DTT), and proteins were separated with a gradient from 100% low-salt buffer to 100% high-salt buffer (20 mM Tris-HCl pH 8, 1 M NaCl, 1 mM DTT) over 176 mL at a flow rate of 4 mL/min. Fractions were evaluated by SDS-PAGE. The DddA11 fractions, free of other contaminants, were collected, buffer-exchanged once with DddA storage buffer (20 mM Tris-HCl pH 7.5, 200 mM NaCl, 1 mM DTT, 5% (w/v) glycerol) using a 10 kDa concentrator, and stored at −80° C.

TDAC-seq DddA11 reaction

1×10⁶ cells were harvested by centrifugation at 300×g for 5 min. The cell pellet was resuspended in 500 μL of PBS, gently pipetted to wash, and centrifuged again at 300×g for 5 min at 4° C. The cell pellet was resuspended in 500 μL of cold lysis buffer (29.5 mM Tris-acetate, 59 mM potassium acetate, 8.9 mM magnesium acetate, 0.09% NP-40, 14.3% dimethylformamide, 10% PBS), and incubated at room temperature for 10 min, followed by incubation at 4° C. for 5 min. After incubation, the cell pellet was centrifuged at 300×g for 5 min at 4° C., and the supernatant was discarded. The cell pellet was then washed with 500 μL of wash buffer (28.05 mM Tris-acetate, 56.1 mM potassium acetate, 8.5 mM magnesium acetate, 0.085% NP-40, 13.6% dimethylformamide, 10% PBS, 20% DddA storage buffer), centrifuged at 300×g for 5 min at 4° C., and the wash buffer was removed. The activation buffer (28.05 mM Tris-acetate, 56.1 mM potassium acetate, 8.5 mM magnesium acetate, 0.085% NP-40, 13.6% dimethylformamide, 10% PBS) was prepared by adding purified DddA11-MBP to achieve a final enzyme concentration of 3-10 μM. The cell pellet was resuspended in 50 μL of activation buffer by gentle pipetting, and the reaction mixture was incubated at 37° C. for 2 h with shaking at 300 rpm. When MGYFPDa829 was used instead of DddA11, we used a final concentration of 3 μM and incubated the reaction at 37° C. for 3 minutes with shaking at 300 rpm. Buffer volume was reduced proportionally if fewer cells were used, with reactions conducted for as few as 2×10⁵ cells. When UGI was added in the reaction, we added 1 μL of UGI (NEB M0281) and 1.25 μL of 10× UGI reaction buffer to a 12.5 μL reaction containing 2.5×10⁵ cells.

To isolate genomic DNA (gDNA), a 50 μL reaction was quenched by adding 5 μL of 10% SDS to a final concentration of 0.5% SDS, along with 35 μL of nuclease-free water and 10 μL of Proteinase K (10 mg/mL). The solution was incubated at 56° C. for 10 min. gDNA was isolated using a Zymo genomic DNA extraction kit (D4064), and the concentration was measured using a NanoDrop microvolume spectrophotometer.

### TDAC-Seq Library Preparation

To minimize primer binding bias, we selected primer binding sites in relatively closed regions and used primers containing Y and R in the TC and CC dinucleotide sequences, which are highly mutated sequence contexts by DddA11. 125-500 ng of gDNA was amplified per 25 μL PCR1 reaction (2× KOD One PCR Master Mix (TYB-KMM-101-5PK), 0.3 μM amplicon-specific forward primer, 0.3 μM amplicon-specific reverse primer). The PCR reaction was conducted with an amplicon size range up to 11.5 kb, under the following conditions: 30 cycles at 98° C. for 10 sec, primer annealing temperature for 5 sec, and 68° C. for 15 min. For semi-nested PCR, the PCR2 reaction was set up by adding 1 μL of PCR1 product. PCR2 was conducted under the same conditions as PCR1 but with 3-8 cycles. 1 μL of PCR2 product was used in barcoding PCR3 with the nanopore PCR Barcoding Expansion 1-96 kit (EXP-PBC096), using 2× KOD One PCR Master Mix. This reaction was run for 12 cycles under the following conditions: 98° C. for 10 sec, 60° C. for 5 sec, and 68° C. for 15 min. To increase PCR yield, PCR1 or 2 products could be purified using 1.2× Mag-Bind® TotalPure NGS Kit (Omega Bio-Tek M1378-01), with the entire eluate used for the next PCR. The final amplicons were purified via DNA gel extraction using the Zymoclean Gel DNA Recovery Kit (D4001) and quantified using a Qubit fluorometer. Barcoded amplicons were pooled in desired ratios, and final nanopore libraries were prepared following the Ligation sequencing V14 (SQK-LSK114), followed by sequencing on a PromethION Flow Cell (R10.4.1) with super accuracy (SUP, v4.3.0) base calling model.

### Determining the Optimal Enzyme Concentration for TDAC-Seq

After purifying a new batch of DddA11 enzyme, we optimized the reaction conditions by performing TDAC-seq on wild-type K562 cells treated with varying concentrations of DddA11. The optimal concentration resulted in approximately 10 as the maximal TDAC-seq signal in two control regions: the DSCR4/8 promoter (chr21: 38118238-38127665) and the HS2 locus (chr11: 5279265-5282582) (FIG. 1C). The optimal DddA11 concentration typically ranges from 3 to 10 μM.

### DddA11-Whole Genome Sequencing

gDNA extracted after the DddA11 reaction was fragmented to 10 kb size using Covaris G-tubes (Covaris 520079), and DNA concentration was measured with a Qubit fluorometer. Nanopore DNA libraries were then generated following the Ligation Sequencing V14-PCR Barcoding (SQK-LSK114 with EXP-PBC096) protocol from Oxford Nanopore Technologies. Briefly, after end repair and barcode adapter ligation, barcoding PCR was performed with 2× KOD One PCR Master Mix, under the following conditions: 16 cycles of 98° C. for 10 sec, 60° C. for 5 sec, and 68° C. for 15 min. The PCR product was purified using 0.4× AMPure XP Beads, quantified using a Qubit fluorometer, and pooled with all barcoded libraries in desired ratios. A total of 1 μg of pooled barcoded libraries was prepared, and final nanopore libraries were completed following the Ligation Sequencing V14 protocol (SQK-LSK114) with end-prep and adapter ligation, followed by sequencing on a PromethION Flow Cell (R10.4.1) with super accuracy (SUP, v4.3.0) base calling model.

### DddA11 Reaction on Methylated Versus Unmethylated Genomic DNA

A 12.5 μL DddA11 reaction was prepared as described above, using 500 ng of either methylated or unmethylated human genomic DNA (Zymo Research, D5014). The DNA was incubated with 0.1 μM DddA11 at 37° C. for 10 minutes. Following incubation, the edited genomic DNA was purified using 2× Mag-Bind® TotalPure NGS magnetic beads. TDAC-seq libraries were then prepared as described above.

### Generating Deproteinized DNA Data

gDNA was extracted from K562 cells using the QIAamp DNA Blood Kit (Qiagen 51104), yielding deproteinized DNA. A 12.5 μL DddA11 reaction was prepared as described above, using 5 μg of purified gDNA. The reaction was incubated for 2 h, with shaking at 300 rpm at 37° C. in a ThermoMixer. To quench the reactions, 1.25 μL of 10% SDS was added to each reaction to achieve a final concentration of 0.5% SDS, followed by 8.75 μL of nuclease-free water and 2.5 μL of Proteinase K (10 mg/mL). The mixture was incubated at 56° C. for 10 min, and gDNA was subsequently isolated using the Zymo Genomic DNA Extraction Kit. The edited DNA was amplified for eight distinct amplicons, and the DNA library was prepared and sequenced as previously described. These eight regions were randomly selected from those where we performed TDAC-seq, many of which were selected based on their high chromatin accessibility and nucleosome footprint signals from ATAC-seq.

### Cell Culture and Lentiviral Transduction

K562 was obtained from ATCC (CCL-243); HEK 293T was a gift from Bradley E. Bernstein; GM12878 was a gift from Xiaowei Zhuang; and MOLM-13 was a gift from Matthew D. Shair. All cell lines were authenticated by Short Tandem Repeat profiling (Genetica) and routinely tested for mycoplasma (Sigma-Aldrich). K562 and MOLM-13 were cultured in RPMI-1640 (Gibco) supplemented with 10% FBS (Peak Serum) and GM12878 were cultured in RPMI-1640 (Gibco) supplemented with 15% FBS. HEK293T and were cultured in DMEM (Gibco) supplemented with 10% FBS. All media were supplemented with 100 U/mL penicillin and 100 μg/mL streptomycin (Life Technologies). All cell lines were cultured in a humidified 5% CO₂ incubator at 37° C. Lentivirus was produced by co-transfecting HEK293T cells with pCMV-VSV-G (a gift from Bob Weinberg; Addgene plasmid #8454), psPAX2 (a gift from Didier Trono; Addgene plasmid #12260) and the transfer vector plasmid encoding gene of interest. Transfections were performed using Lipofectamine 3000, following the manufacturers' protocols. Media was exchanged after 6-12 h and the viral supernatant was collected 48 h post-transfection and filtered (0.45 μm). Transduction was carried out by mixing the virus with cells with 8 μg/ml polybrene for K562 and 5 μg/ml polybrene for MOLM-13 then spinfected at 1,800 g for 90 min at 37° C. After 48 h post-transduction, media was changed and puromycin (Thermo Fisher Scientific) selection was carried out for 4 days.

Generation K562 Cell Lines Expressing dCas9 with sgRNA

The pHR-SFFV-dCas9-BFP plasmid was subcloned from pHR-SFFV-dCas9-BFP-KRAB (gift from Stanley Qi and Jonathan Weissman, Addgene #46911). K562 cells were transduced with lentiviral particles carrying the pHR-SFFV-dCas9-BFP plasmid as described above and cells with BFP positive cells were sorted on a MoFlo Astrios EQ cell sorter. The sgRNA sequence targeting the HEK3 locus, GGCCCAGACTGAGCACGTGA (SEQ ID NO: 1), was cloned into pLentiGuide-Puro (gift from Feng Zhang, Addgene plasmid #52953). Lentiviral particles carrying the plasmids were generated as described above, and K562 dCas9-BFP cells were transduced and subsequently selected with puromycin (2 μg/mL).

Human CD34⁺ HSPCs culture and three-phase erythroid differentiation

Human CD34⁺ HSPCs (G-CSF mobilized PBSC-CD34 enriched) from mobilized peripheral blood of healthy adults were obtained from the Cooperative Center of Excellence in Hematology at the Fred Hutchinson Cancer Research Center. All donors provided informed consent for genomic sequencing. The primary HSPCs were thawed into a maintenance medium consisting of a StemSpan II base (StemCell Technologies), CC100 (StemCell Technologies), 50 ng/mL human TPO (Pepro Tech), 35 nM UM171 (StemCell Technologies) and 1% penicillin/streptomycin (Life Technologies) and 1% of L-Glutamine (Life Technologies).

After the maintenance phase, primary human HSPCs were differentiated using the three-phase culture system previously described. First, a base erythroid medium was created by supplementing IMDM (Gibco) with 2% human AB plasma (SeraCare), 3% human AB serum (Life Technologies), 3 U/mL heparin, 10 μg/mL insulin, 200 μg/mL holo-transferrin, and 1% penicillin/streptomycin. From days 1-7 in erythroid media, this base medium was further supplemented with 3 U/mL EPO, 10 ng/ml human SCF, and 1 ng/mL IL-3. From days 7-12, this base medium was further supplemented with 3 U/mL EPO and 10 ng/ml human SCF. After day 12, the base medium was supplemented with 1 mg/mL total of holo-transferrin and 3 U/mL of EPO.

HBG1/2 Cas9 Deletions in CD34⁺ HSPCs with Erythroid Differentiation

The sgRNA-68 sequence targeting HBG promoter is ACTGAATCGGAACAAGGCAA (SEQ ID NO: 2). Oligos containing the sgRNA sequences were cloned into pLentiGuide-Puro (Addgene plasmid #52953). Lentiviral particles carrying the plasmids were generated as described above. Cell supernatant was collected and filtered through a 0.45 mM membrane and concentrated using Lenti-X Concentrator (Takara Bio, 631232).

Human CD34+ cells were cultured in hematopoietic stem cell (HSC) expansion media consisting of StemSpan SFEM II (StemCell Technologies, 02690) supplemented CC100 cytokine cocktail (StemCell Technologies, 02690), 50 ng/mL human TPO (PeproTech, 300-18), 1% L-Glutamine (Gibco), 1% penicillin/streptomycin (Gibco), and 35 nM UM171. After a one-day maintenance phase, lentivirus was pre-coated on plates with Retronectin (Takara Bio, T110A) and cells were transduced by spinfection at 800 g for 1.5 h at 37° C. and cultured for 3 days.

To generate double stranded DNA breaks with sgRNA-68, Alt-R Cas9 protein (IDT) was transfected into cells via electroporation using NEON system (Thermo Fisher Scientific) with the following conditions: 1600 V, 10 ms, 3 pulses. Following electroporation, cells were transferred into pre-warmed HSC media and placed in culture for 2 days.

For erythroid differentiation, CD34⁺ cells were differentiated in vitro toward the erythroid lineage using an adaptation of the culture method. Briefly, cells were cultured in erythroid differentiation medium (EDM), consisting of Iscove's Modified Dulbecco's Medium (IMDM) (Gibco), 330 mg/ml human holotransferrin (Sigma, T4132), 10 mg/ml recombinant human insulin (Sigma, 19278), 2 IU/ml heparin (Sigma, H3149), 5% human AB serum (Research Products Internation, H68250), 2.5 U/ml human erythropoietin (PeproTech, 100-064), 1% glutamine (Gibco) and 1% penicillin/streptomycin (Gibco). EDM was further supplemented with 1.38 mM hydrocortisone (StemCell Technologies, 07925), 100 ng/ml human SCF (PeproTech, 300-07), 5 ng/ml human IL-3 (PeproTech, 200-03) and 1 mg/ml puromycin (Gibco) for cell selection. Cells were grown in humidified incubators at 37° C. and 5% CO₂. Cells were harvested for cell staining, ATAC-seq, and TDAC-seq on Days 5 post-differentiation induction.

### Staining for Flow Cytometry

Human CD34+ cells were stained at day 5 of erythroid differentiation for 2-phase culture system. For staining of CD71 and CD235a surface markers, cells were washed once with PBS and stained with 1:50 dilution of anti-CD71-BV711 (BD Biosciences, Clone M-A712, Cat #563767) and anti-CD235a-PE (eBioscience, Clone HIR2 (GA-R2), Cat #12-9987-82) for 30 min at 4° C. For 3-phase culture, we used anti-CD71-BV421 (Biolegend, Clone CY1G4, Cat #334122) and anti-CD235a-APC-Cy7 (Biolegend, Clone HI264, Cat #349116). For intracellular staining of HbF, cells were washed, fixed with fixation buffer (Biolegend, 420801) and permeabilized with intracellular staining permeabilization wash buffer (Biolegend, 421002). Cells were then incubated with 1:40 dilution of anti-HbF-PE (Life Technologies, Clone HBF-1, MHFH04) or anti-HbF-APC (Life Technologies, Clone HBF-1, MHFH05) for 20 min at 4° C. After staining, the cells were washed twice with wash buffer, resuspended in staining buffer. Data acquisition was performed on ACEA Novocyte flow cytometer (Agilent) using NovoExpress software (v1.6.1). Data were analyzed with FlowJo (v.10.10).

### ABE8e Purification

ABE8e was purified as previously described. Briefly, the plasmid (Addgene plasmid #161788) was transformed into BL21 (DE3) pLysS E. coli cells, and cultures were maintained in TB media with 25 mg/mL kanamycin. Following saturated overnight culture, 2 L of TB media with 25 mg/mL kanamycin was inoculated and grown at 37° C. until the OD600 reached 1.5. To induce protein expression, the culture was supplemented with 30% L-rhamnose (Sigma-Aldrich, R3875-100G) to a final concentration of 0.8%, then incubated at 18° C. for 24 h with shaking. Cells were harvested by centrifugation at 4000×g for 35 min.

Cell pellets from 1 L of culture were lysed by resuspending in 30 mL of cold bacterial lysis buffer containing 20 mM HEPES, pH 7.5, 2 M NaCl, 10% glycerol, 1 mM TCEP (added fresh), and two tablets of Roche EDTA-free complete protease inhibitor cocktail. DNase I (75 U) was added, and the suspension was sonicated (10 min, 60% amplitude, 10 s on/off cycles) on ice. The lysate was clarified by centrifugation at 25,000 g for 30 min at 4° C. The supernatant was incubated with 0.75 mL of pre-washed His60 nickel resin (Takara Bio) at 4° C. for 1 h to facilitate protein binding. The resin was washed sequentially with 10 mL of water and 10 mL of lysis buffer supplemented with 1 mM TCEP. After washing, the resin was loaded into a column, and the column was washed with 100 mL of wash buffer (20 mM HEPES, pH 7.5, 2 M NaCl, 10% glycerol, 1 mM TCEP, 25 mM imidazole). Proteins were eluted with 4 mL of elution buffer (20 mM HEPES, pH 7.5, 10% glycerol, 1 mM TCEP, 500 mM imidazole), collecting fractions after 10 min of incubation. To further purify the protein, the elution fractions were diluted with 50 mL of low-salt buffer (20 mM HEPES, pH 7.5, 10% glycerol, 1 mM TCEP) and loaded onto a HiTrap SP HP cation exchange column (Cytiva, 17115101) connected to FPLC system. The column was equilibrated in low-salt buffer, and proteins were separated with a gradient from 100% low-salt buffer to 80% high-salt buffer (20 mM HEPES, pH 7.5, 2 M NaCl, 10% glycerol, 1 mM TCEP) over 50 mL at a flow rate of 5 mL/min. Fractions were collected and analyzed on a 4-12% Bis-Tris gradient gel using MOPS buffer. Fractions containing the target protein were pooled and concentrated using an Amicon Ultra-0.5 Centrifugal Filter Unit (Sigma, UFC5100BK), flash-frozen, and stored at −80° C.

TDAC-Seq Integrated with CRISPR Perturbation

The sgRNA sequences targeting the HOXA7/9 CTCF/YY1 binding site are as follows: HOXA_sgRNA-1: GCCATCTGCTGGCCGCCGTT (SEQ ID NO: 3), HOXA_sgRNA-2: ACCAAACGGCGGCCAGCAGA (SEQ ID NO: 4), HOXA_sgRNA-3: GGAGCCACACTGCCATCTGC (SEQ ID NO: 5), HOXA_sgRNA-4: GCGGCCAGCAGATGGCAGTG (SEQ ID NO: 6). Oligos containing the sgRNA sequences were cloned into pLentiGuide-Puro (gift from Feng Zhang, Addgene plasmid #52953). For the pooled HS2 library, each sgRNA was pooled at equal ratios. Lentiviral particles carrying the plasmids were generated as described above and titered according to published procedures.³⁹ For the HS2 screen, K562 cells (2.5×10⁵) were transduced at a multiplicity of infection <0.3 and subsequently selected with puromycin. For individual sgRNA transduction experiments, virus titration was omitted. After 5 days of puromycin selection (1 μg/mL puromycin for MOLM-13 and 2 μg/mL puromycin for K562), cells were electroporated with Cas9 or ABE8e. 4×10⁵ cells were prepared for electroporation by washing twice in PBS, and cells were resuspended in Resuspension Buffer R at a concentration of 2×10⁷ cells/mL. 37.2 μM Alt-R Cas9 protein (IDT) was prepared by diluting 62 μM stock with Buffer R. For each electroporation, 1 μL of diluted Cas9 or purified ABE8e (14 μM) was mixed with 11 μL of cell suspension and electroporated at 1000 V with 50 ms pulse width for 1 pulse using the Neon Transfection System 10 μL kit (Thermo Fisher, MPK1025). Electroporation was performed twice, and cells were transferred into pre-warmed RPMI medium with 10% FBS (without antibiotics). After 6 days post-electroporation, TDAC-seq was performed and samples were sequenced as described above. For single guide experiments, 200-400 ng of gDNA was amplified in PCR1, and for the pooled screen, the amount of amplified gDNA was increased proportionally to the library size by increasing the number of PCR1 reactions.

### GF11B Enhancer CRISPR Cas9 Perturbation in CD34⁺ HSPCs

Oligos containing the sgRNA sequences tiling the GF11B enhancer were synthesized as an oligonucleotide pool (Twist Biosciences) and cloned into the pLentiGuide-Puro vector (Addgene plasmid #52953). Lentiviral particles carrying the pLentiGuide-Puro GF11B library DNA were generated as described above. Cell supernatant was collected and filtered through a 0.45 mM membrane and concentrated using Lenti-X Concentrator (Takara Bio, 631232).

Cryopreserved human CD34⁺ cells were obtained from the mobilized peripheral blood of healthy human donors (Fred Hutchinson Cancer Center). Cells were cultured in hematopoietic stem cell (HSC) expansion media consisting of StemSpan SFEM II (StemCell Technologies, 02690) supplemented CC100 cytokine cocktail (StemCell Technologies, 02690), 50 ng/mL human TPO (PeproTech, 300-18), 1% L-Glutamine (Gibco), 1% penicillin/streptomycin (Gibco), and 35 nM UM171. After a one-day maintenance phase, lentivirus was pre-coated on plates with Retronectin (Takara Bio, T110A) and cells were transduced by spinfection at 800 g for 1.5 hours at 37° C. and cultured for 3 days.

Alt-R Cas9 protein (IDT) was transfected into cells via electroporation using NEON system (Thermo Fisher Scientific) with the following conditions: 1600 V, 10 ms, 3 pulses. Following electroporation, cells were transferred into pre-warmed HSC media and placed in culture for 2 days. The culture media was then replaced with HSC media supplemented with 1 μg/mL puromycin (Gibco) for selection. Cells were grown in humidified incubators at 37° C. and 5% CO₂. After 4 days of puromycin selection, cells were harvested for TDAC-seq. 5×10⁶ cells were used per TDAC-seq replicate.

### ATAC-Seq

Cells were washed twice with cold PBS and resuspended in PBS at a density of 2×10⁶/mL, ensuring a final reaction cell count of approximately 1×10⁴ cells per 5 μL. Next, 5 μL of cells in PBS were mixed 42.5 μL of transposition buffer (38.8 mM Tris-acetate, 77.6 mM potassium acetate, 11.8 mM magnesium acetate, 0.12% NP-40, 18.8% dimethylformamide). The reaction was incubated at room temperature for 10 min, followed by the addition of 2.5 μL of assembled Tn5 transposase, and then incubated at 37° C. for 30 min in a ThermoMixer set to 300 rpm. DNA was subsequently purified with the Qiagen MinElute PCR Purification Kit (Qiagen) and minimally amplified for sequencing, following established protocols. Final libraries were purified again with the MinElute PCR Purification Kit (Qiagen) and sequenced on a NextSeq 550 or Novaseq SP.

After removing adapters using NGmerge, the ATAC-seq data was aligned using bowtie2 (v.2.5.4), processed using samtools (v.1.19.2) and duplicates were removed by using picard MarkDuplicates (v.3.1.1). Bam files were converted into fragments files by using bedtools bamtobed (v.2.31.1). Peak calling was performed using MACS2 (v.2.2.9.1). Downstream analysis was performed using R 4.4.1 with the following packages: SummarizedExperiment (v.1.34.0), GenomicRanges (v.1.56.2), GenomeInfoDb (v.1.40.1), ggplot2 (v.3.5.1), data.table (v.1.16.2), cladoRcpp (v.0.15.1), stringr (v.1.5.1), pbmcapply (v.1.5.1), rtracklayer (v.1.64.0), TxDb.Hsapiens.UCSC.hg38.knownGene (v.3.18.0). The genomic region of interest was divided into 1 bp bins and the number of Tn5 fragment ends overlapping with each bin was quantified as the Tn5 insertion density. Then the insertion density track was smoothed by a 250 bp window to obtain the final coverage track. The coverage tracks were exported to bigwig format and visualize in IGV.

### Quantitative Reverse-Transcription PCR

Cells were harvested for total RNA isolation using the RNeasy Mini Kit (Qiagen). cDNA synthesis was performed with the Applied Biosystems™ High-Capacity cDNA Reverse Transcription Kit (Applied Biosystems™ 4368814). Quantitative reverse transcription PCR (RT-qPCR) was conducted using SYBR™ Select Master Mix on the CFX Connect Real-Time PCR Detection System (Bio-Rad) with oligonucleotide primers. Results were calculated as the fold-change in mRNA expression of the gene of interest, normalized to GAPDH expression, using the method ΔΔCt. GraphPad Prism (v.10.4.0) was used to generate the bar plots.

### Targeted Cas9-Based Enrichment of the HS2 Locus

We followed a previously established protocol. Briefly, one million K562 cells were s ubjected to in situ GpC methylation using M.CviPI (NEB, M0227L), and high-molecular-wei ght (HMW) genomic DNA was extracted.

A total of four different RNP complexes were engineered to target the flanking region s of the HS2 locus, spanning approximately 3.4 kb. sgRNA sequences were designed and ord ered (CAAGCTATAAACTTTTCCTCAGG (SEQ ID NO: 7), CAGACTAAACTTGAAATA TGTGG (SEQ ID NO: 8), CATGCACACTGGTCAAAAGTAGG (SEQ ID NO: 9), GGAAA CTTTGTTGTCAGACCCGG (SEQ ID NO: 10)). Double-stranded DNA was synthesized usi ng the EnGen sgRNA Synthesis Kit (NEB, E3322S) and purified using the Agencourt RNAC lean XP system (Beckman Coulter). RNP complexes were assembled, and DNA dephosphory lation was conducted as previously described, with the following modifications. 5 μg of HM W genomic DNA were dephosphorylated with 10 μl of rSAP (NEB, M0371S) and 16 μl of 1 0× rCutSmart buffer (NEB, B6004S) for 30 minutes at 37° C., followed by heat inactivation at 65° C. for 5 minutes. The DNA was then purified using 0.7× volume of PacBio AMPure PB b eads (Pacific Biosciences, 100-265-900). Briefly, AMPure PB beads were added to the depho sphorylation reaction, followed by a 15-minute incubation at room temperature and magnetic separation for 10 minutes. The beads were washed twice with 80% ethanol, and DNA was elu ted in 40 μl of nuclease-free water at 37° C. The dephosphorylated DNA sample was combin ed with the pooled RNP complexes for CRISPR digestion. After dA-tailing, a nanopore adapt er was ligated using the ONT LSK114 kit. 1 μg of the final DNA library was loaded onto a Pr omethION flow cell.

### DddA11-Whole Genome Sequencing Analysis

Nanopore sequencing data were aligned using Minimap2 (v.2.28). Duplicate reads were removed, and base-specific mutation counts were extracted using Samtools (v.1.21) mpileup. Mutation rates at each cytosine were calculated, saved as a bedGraph file, and converted to a BigWig file using ucsc-bedgraphtobigwig (v.472). The distribution of cytosine deamination fractions around CTCF binding sites or TSS was analyzed and plotted using computeMatrix and plotProfile from deepTools (v.3.5.5). All final figures in this study were generated using Adobe Illustrator 2022 (v.26.0.3).

Detection of DddA11 Base Edits from TDAC-Seq

Nanopore sequencing data were aligned using the Minimap2 (v.2.28) and analyzed using Python (3.10.14) with the following packages: mappy (v.2.28), NumPy (v.1.26.4), Biopython (v.1.83), SciPy (v.1.14.0), umi_tools (v.1.1.5), pandas (v.2.2.2), matplotlib (v.3.8.4), hmmlearn (v.0.3.3), seaborn (v.0.13.2), pyranges (v.0.1.2), dna-features-viewer (v.3.1.3), liftover (v.1.2.2), pyBigWig (v.0.3.22), and crispresso2 (v.2.3.1). Reads with larger than 500 bp unaligned regions compared to the full amplicon on either end of the read were removed. Single base mutations and indels were extracted using the cs tag after alignment. CpG sites were masked from mutation calling to prevent bias introduced by endogenous CpG methylation. Only C-to-T and G-to-A mutations were recorded as potential DddA11 edits. Reads with more C-to-T than G-to-A mutations were assigned to the top strand of the template and vice versa. TDAC-seq signals were compared with published ATAC-seq and ChIP-seq data.

### Computing DddA11 Sequence Bias

DddA11 sequence bias was estimated using DddA11 edit rate on naked DNA with a k-mer model (k=3). k-mers with center bases being A and T were assigned with a bias of 0. Of the eight regions amplified from DddA11-treated naked DNA, four regions at chrX: 48798803-48803519, chr8: 127733151-127737826, chr3: 141366125-141371787, and chr7: 107741812-107745536 were randomly selected for model fitting, and regions at chr1: 170530445-170533865, chr21: 38118238-38127665, chr5: 180324459-180334556, and chr7: 27158522-27163197, were selected for model testing.

### Calculating DddA11 Footprints

DddA11 footprints were computed either on individual DNA molecules or aggregated reads. For each single base-pair position of interest. The sum of DddA11 edits were computed for a center region with a given radius r, as well as two flanking regions with a diameter of r. Similarly, the sum of DddA11 sequence bias was computed for the same center and flanking regions. Then the depletion of DddA11 edits at the center was calculated using a left-tailed center-versus-flank binomial test using bias_center/(bias_center+bias_flank) as the probability p of binomial distribution. A pseudo-count was added to prevent division of zero. Testing was performed for the left and right flank separately, and the least significant p-value was kept as the final p-value. Eventually, the −log₁₀ (p-value) was used as the footprint score. DddA11 footprints were compared with published MNase-seq and ChIP-seq data.

### Analysis of Pooled CRISPR Screen Data

CRISPR deletions were detected during alignment of nanopore sequencing reads by parsing the cs tag. Deletions larger than 5 bp were kept as CRISPR deletions. Alternatively, for samples of the hybrid HBG locus treated with sgRNA-68, trimmed sequencing reads were input to CRISPResso with the hybrid locus as the reference sequence, and 4928 bp was further subtracted from the outputted annotations to adjust for the large deletion. To estimate the effect of each guide, we compared wild type reads (selected as background reads) to those with deletions at the site of interest and without any deletion outside this window (selected as foreground reads). The foreground and background reads were first down-sampled to the same read number, then deduplicated. Then the average DddA11 accessibility, as well as footprints were compared between the foreground and background. Here, the comparison was performed separately for the C-to-T strand and G-to-A strand and then averaged to prevent any bias introduced by imbalanced strand sampling. Additionally, DddA11 signal within the window of interest is masked to be 0 to prevent false positive differences resulting from the CRISPR deletion itself. For differential footprints, footprints were first calculated on single DNA molecules before aggregating to obtain the final result.

Identification of Minimal Regulatory Element from Diverse CRISPR Deletions

We considered whether diverse CRISPR deletion genotypes share a common mechanism by deleting the same minimal regulatory element. To nominate such a regulatory element, we systematically considered all 1-15 bp (deletion window) subsequences. For each candidate subsequence, we grouped reads that contain a deletion spanning the entire window and where the deletion length is at most 10 bp longer than the window. All other reads were assigned to a second group. Welch's t-test was performed to compare the number of DddA11 edits per read in the two groups, and we used the p-value to reflect the quality of using the candidate deletion window to group reads, similar to traditional clustering algorithms. Windows with high purity correspond to a subsequence whose deletion is necessary and sufficient for an effect on DddA11 accessibility.

### Analysis of Pooled ABE Screen Data

ABE base edits were detected during alignment of nanopore sequencing reads by parsing the cs tag. Adenine bases in positions +3 to +9 of the sgRNA were used for analysis. The ratio of editing rate at each adenine in the presence and absence of ABE treatment was used to threshold candidate adenine positions (threshold is dependent on the dataset), and the strand of A-to-G edits was examined to ensure the correct adenines were selected. Instead of comparing sgRNAs, the effect of editing at each individual adenine was estimated by comparing reads where the adenine of interest was edited with reads without any edits throughout. Then edited and unedited reads were down-sampled to the same number, deduplicated, and used to compare DddA11 accessibility.

### Comparison to TF Binding Sites

High confidence TF binding sites were obtained from UniBind. The individual TF binding events were superimposed onto the genomic region of interest. Overlapping TF binding site annotations were merged. The final TF binding event list was visualized using the DNA Features Viewer Python package.

### Sequencing Read Deduplication

To reduce the effect of PCR bias, we identified and removed sequencing reads from PCR duplicates using the pattern of DddA11 edits across the amplicon. Because the amplicon is large, it is unlikely for any pair of cells to receive C·G-to-T. A edits in the exact same or extremely similar positions, so such reads are attributed to PCR duplication. As such, for each read, we extracted a binary vector indicating whether each C·G in the reference has been edited to T·A and used this as a unique molecular identifier (UMI). We filtered out reads that did not have enough edits (less than 2% C·G-to-T·A) to robustly differentiate unique reads. UMIs are grouped together using UMI-tools if their hamming distance is less than 2% of the UMI length. Finally, for each group, the read with the most common UMI is kept.

### Calling Deaminase-Accessible DNA Sequences (DADs)

For each read, we identified regions with a high density of DddA11 edits, termed deaminase-accessible DNA sequences (DADs). To account for the enzyme's substrate preferences (i.e. it only edits cytosines, which naturally occur at variable densities in the genome, and its editing efficiency is strongly determined by the preceding nucleotide), we used a hidden Markov model based on the hmmlearn implementation but with custom emission probabilities. In the model, each position is in either an open region (DAD) or a closed region. In the former case, the C·G-to-T. A editing rate is higher and depends on sequence context because such events are attributed to DddA11 editing. Meanwhile, in the latter case, the editing rate is lower because such events are attributed to sequencing error. The model parameters were obtained by fitting the sequencing data for each TDAC-seq locus (FIG. 1E) and selecting a representative, well-performing model. DADs shorter than 70 bp were filtered out.

1. A method for identifying a therapeutic genomic target sequence, the method comprising:
(a) perturbing a genomic locus using a gene editing system;
(b) deaminating one or more nucleotides of the genomic locus using a deaminase to produce a pattern of deamination at the genomic locus; and
(c) identifying a therapeutic genomic target sequence based on the pattern of deamination at the genomic locus.

2. The method of claim 1, wherein the gene editing system is selected from CRISPR-Cas systems, TALEN (Transcription Activator-Like Effector Nuclease) systems, ZFN (Zinc Finger Nuclease) systems, and meganuclease (homing endonuclease) systems.

3. The method of claim 2, wherein the gene editing system is a CRISPR-Cas system.

4. The method of claim 3, wherein the CRISPR-Cas system comprises a guide RNA (gRNA) and a Cas nuclease.

5. The method of claim 4, wherein the guide RNA comprises the nucleotide sequence of any one of SEQ ID NOs: 1-10.

6. The method of claim 3, wherein the CRISPR-Cas system comprises a deactivated Cas (dCas) nuclease.

7. The method of claim 6, wherein the CRISPR-Cas system further comprises a base editor.

8. The method of claim 3, wherein the CRISPR-Cas system comprises a Cas nickase (nCas).

9. The method of claim 8, wherein the CRISPR-Cas system further comprises a reverse transcriptase.

10. The method of claim 1, wherein the deaminase is a double-stranded DNA deaminase.

11. The method of claim 10, wherein the double-stranded DNA deaminase is double-stranded DNA deaminase toxin A (DddA), DddB, DddSs, DddDd-5, MGYFPDa829, or CseDa01, and optionally wherein the DddA is DddA11.

12. The method of claim 1, wherein the genomic locus comprises a cis-regulatory element.

13. The method of claim 1, wherein (c) comprises amplifying the genomic locus to produce amplicons of the genomic locus.

14. The method of claim 13, wherein (c) further comprises sequencing the amplicons of the genomic locus.

15. The method of claim 14, wherein sequencing of the amplicons comprises long-read sequencing.

16. A method for screening for guide RNAs targeting regulatory elements that modulate chromatin accessibility, the method comprising:
(a) perturbing genomic loci using a CRISPR-Cas system comprising a pooled library of guide RNAs;
(b) deaminating one or more nucleotides of the respective genomic loci using a deaminase to produce a pattern of deamination at the respective genomic loci; and
(c) identifying guide RNAs targeting regulatory elements that modulate chromatin accessibility based on the pattern of deamination at the respective genomic loci.

17-27. (canceled)

28. A kit, comprising a deaminase or a nucleic acid encoding the deaminase, a gene editing system, and materials and/or reagents for executing the method of claim 1.

29-50. (canceled)

51. The method of claim 4, wherein the Cas nuclease is selected from Cas9 enzymes and Cas12 enzymes.

52. The method of claim 7, wherein the base editor is selected from selected from cytosine base editors and adenine base editors.

53. The method of claim 13, wherein the amplifying is performed using PCR (polymerase chain reaction).


SEQUENCE LISTING (46 sequences)

SEQ ID NO: 1 - RNA, 20 nt, synthetic construct
  source 1..20: mol_type = other RNA
ggcccagactgagcacgtga

SEQ ID NO: 2 - RNA, 20 nt, synthetic construct
  source 1..20: mol_type = other RNA
actgaatcggaacaaggcaa

SEQ ID NO: 3 - RNA, 20 nt, synthetic construct
  source 1..20: mol_type = other RNA
gccatctgctggccgccgtt

SEQ ID NO: 4 - RNA, 20 nt, synthetic construct
  source 1..20: mol_type = other RNA
accaaacggcggccagcaga

SEQ ID NO: 5 - RNA, 20 nt, synthetic construct
  source 1..20: mol_type = other RNA
ggagccacactgccatctgc

SEQ ID NO: 6 - RNA, 20 nt, synthetic construct
  source 1..20: mol_type = other RNA
gcggccagcagatggcagtg

SEQ ID NO: 7 - RNA, 23 nt, synthetic construct
  source 1..23: mol_type = other RNA
caagctataaacttttcctcagg

SEQ ID NO: 8 - RNA, 23 nt, synthetic construct
  source 1..23: mol_type = other RNA
cagactaaacttgaaatatgtgg

SEQ ID NO: 9 - RNA, 23 nt, synthetic construct
  source 1..23: mol_type = other RNA
catgcacactggtcaaaagtagg

SEQ ID NO: 10 - RNA, 23 nt, synthetic construct
  source 1..23: mol_type = other RNA
ggaaactttgttgtcagacccgg

SEQ ID NO: 11 - protein, 8 aa, synthetic construct
GGGSGGGS

SEQ ID NO: 12 - DNA, 51 bp, synthetic construct
  source 1..51: mol_type = other DNA
gcctcttgcatggagccacactgccatctgctggccgccgtttggtactgc

SEQ ID NO: 13 - DNA, 20 bp, synthetic construct
  source 1..20: mol_type = other DNA
acaccctaagcctcagcatt

SEQ ID NO: 14 - DNA, 14 bp, synthetic construct
  source 1..14: mol_type = other DNA
acaccctaagcatt

SEQ ID NO: 15 - DNA, 20 bp, synthetic construct
  source 1..20: mol_type = other DNA
acaccctaagactcagcatt

SEQ ID NO: 16 - DNA, 26 bp, synthetic construct
  source 1..26: mol_type = other DNA
acaccctaagcctcagactcagcatt

SEQ ID NO: 17 - DNA, 19 bp, synthetic construct
  source 1..19: mol_type = other DNA
acaccctaagctcagcatt

SEQ ID NO: 18 - DNA, 14 bp, synthetic construct
  source 1..14: mol_type = other DNA
acaccctcagcatt

SEQ ID NO: 19 - DNA, 18 bp, synthetic construct
  source 1..18: mol_type = other DNA
acaccctaactcagcatt

SEQ ID NO: 20 - DNA, 12 bp, synthetic construct
  source 1..12: mol_type = other DNA
acactcagcatt

SEQ ID NO: 21 - DNA, 40 bp, synthetic construct
  source 1..40: mol_type = other DNA
acaccctaagcctcagcatgactcatcatgactcagcatt

SEQ ID NO: 22 - DNA, 30 bp, synthetic construct
  source 1..30: mol_type = other DNA
acaccctaagcctcatcatgactcagcatt

SEQ ID NO: 23 - DNA, 34 bp, synthetic construct
  source 1..34: mol_type = other DNA
acaccctaagcatgactcatcatgactcagcatt

SEQ ID NO: 24 - DNA, 33 bp, synthetic construct
  source 1..33: mol_type = other DNA
acacctcagcatgactcatcatgcatcagcatt

SEQ ID NO: 25 - DNA, 27 bp, synthetic construct
  source 1..27: mol_type = other DNA
acaccctaagcctcatgactcagcatt

SEQ ID NO: 26 - DNA, 34 bp, synthetic construct
  source 1..34: mol_type = other DNA
acaccctcagcatgactcatcatgactcagcatt

SEQ ID NO: 27 - DNA, 30 bp, synthetic construct
  source 1..30: mol_type = other DNA
acaccctaagactcatcatgactcagcatt

SEQ ID NO: 28 - DNA, 15 bp, synthetic construct
  source 1..15: mol_type = other DNA
acatgactcagcatt

SEQ ID NO: 29 - DNA, 40 bp, synthetic construct
  source 1..40: mol_type = other DNA
ttttttgccatctgccctgtgagcatcctgctggggaccc

SEQ ID NO: 30 - DNA, 40 bp, synthetic construct
  source 1..40: mol_type = other DNA
ttttttgccatctgccctgtgggcatcctgctggggaccc

SEQ ID NO: 31 - DNA, 40 bp, synthetic construct
  source 1..40: mol_type = other DNA
ttttttgccatctgccctgtaagcatcctgctggggaccc

SEQ ID NO: 32 - DNA, 40 bp, synthetic construct
  source 1..40: mol_type = other DNA
ttttttgccatctgccctgtaagcatcctgccggggaccc

SEQ ID NO: 33 - DNA, 40 bp, synthetic construct
  source 1..40: mol_type = other DNA
ttttttgccatccgccctgtaagcatcctgctggggaccc

SEQ ID NO: 34 - DNA, 40 bp, synthetic construct
  source 1..40: mol_type = other DNA
ttttttgccatctgccctgtaggcatcctgctggggaccc

SEQ ID NO: 35 - DNA, 40 bp, synthetic construct
  source 1..40: mol_type = other DNA
ttttttgccatccgccccgtaagcatcctgctggggaccc

SEQ ID NO: 36 - DNA, 40 bp, synthetic construct
  source 1..40: mol_type = other DNA
ttttttgccatctgccccgtaagcatcctgctggggaccc

SEQ ID NO: 37 - DNA, 40 bp, synthetic construct
  source 1..40: mol_type = other DNA
acaccctaagcctcagcatgacccatcatgactcagcatt

SEQ ID NO: 38 - DNA, 40 bp, synthetic construct
  source 1..40: mol_type = other DNA
acaccctaagcctcagcatgacccaccacgactcagcatt

SEQ ID NO: 39 - DNA, 40 bp, synthetic construct; identical to SEQ ID NO: 21
  source 1..40: mol_type = other DNA

SEQ ID NO: 40 - DNA, 40 bp, synthetic construct
  source 1..40: mol_type = other DNA
acaccctaagcctcagcatgactcaccacgactcagcatt

SEQ ID NO: 41 - DNA, 40 bp, synthetic construct
  source 1..40: mol_type = other DNA
acaccctaagcctcagcatgactcatcacgactcagcatt

SEQ ID NO: 42 - DNA, 40 bp, synthetic construct
  source 1..40: mol_type = other DNA
acaccctaagcctcagcatgactcaccatgactcagcatt

SEQ ID NO: 43 - DNA, 23 bp, synthetic construct
  source 1..23: mol_type = other DNA
ccggcctgagacttatcactggc

SEQ ID NO: 44 - DNA, 37 bp, synthetic construct
  source 1..37: mol_type = other DNA
tcctccaaccacctcaccctgggtgattcagcaaatc

SEQ ID NO: 45 - DNA, 34 bp, synthetic construct
  source 1..34: mol_type = other DNA
gtgcagagctggccaaagaggaagtgccacggcc

SEQ ID NO: 46 - DNA, 24 bp, synthetic construct
  source 1..24: mol_type = other DNA
cttggagggaaaaagagaagggca